(19) United States
(12) Patent Application Publication
(10) Pub. No.: US 2007/0117852 A1
(43) Pub. Date: May 24, 2007

(54) (Hetero)cyclyl carboxanilides for controlling harmful fungi

(76) Inventors: Markus Gewehr, Kastellaun (DE); Bernd Muller, Frankenthal (DE); Thomas Grote, Wachenheim (DE); Wassilios Grammenos, Ludwigshafen (DE); Jordi Tormo i Blasco, Laudenbach (DE); Anja Schwogler, Mannheim (DE); Joachim Rheinheimer, Ludwigshafen (DE); Carsten Blettner, Mannheim (DE); Peter Schafer, Ottersheim (DE); Frank Schieweck, Hessheim (DE); Oliver Wagner, Neustadt (DE); John-Bryan Speakman, Bobenheim (DE); Jan Rether, Kaiserslautern (DE); Siegfried Strathmann, Limburgerhof (DE); Reinhard Stierl, Freinsheim (DE)

Correspondence Address: BIRCH STEWART KOLASCH & BIRCH, PO BOX 747, FALLS CHURCH, VA, 22040-0747, US

(21) Appl. No.: 10/582,040

(22) PCT Filed: Dec. 22, 2004
(86) PCT No.: PCT/EP04/14622

§ 371 Date: Jun. 7, 2006

(30) Foreign Application Priority Data
Dec. 23, 2003 (DE) 103 61 005.7

(51) Int. Cl. A01N 43/56; A01N 43/40; A01N 43/78; C07D 213/24

(52) U.S. Cl. 514/357; 514/365; 514/406; 548/190; 548/370.1; 546/334

(57) ABSTRACT

The present invention relates to (hetero)cyclylcarboxanilides of the formula I, 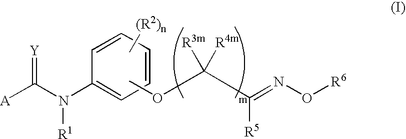
in which n is 0, 1, 2, 3 or 4; and m is 1, 2 or 3, Y is oxygen or sulfur; A is unsubstituted or substituted phenyl or is an at least monounsaturated, unsubstituted or substituted 5- or 6-membered heterocycle, R¹, R², R^{3m}, R^{4m}, R⁵ and R⁶ are as defined in claim 1, and their agriculturally useful salts. Moreover, the present invention relates to the use of the (hetero)cyclylcarboxanilides of the formula I and their agriculturally useful salts as fungicides, and to crop protection compositions comprising these compounds.

The present invention relates to (hetero)cyclic carboxanilides having an oxime ether function, and to their use for controlling harmful fungi.

WO 02/08195 describes fungicidally active 1,3-dimethyl-5-fluoropyrazole-4-carboxanilides having, in the 2-position of the phenyl ring, a phenyl group having an oxime ether group. WO 02/08197 discloses hetarylanilides of a similar structure. WO 98/03500 describes carbanilides which may, inter alia, have an oxime aryl ether group on the phenyl ring. However, examples of these are not given.

However, in particular at low application rates, the (heteroaryl)carboxanilides described in these publications are not entirely satisfactory. It is an object of the present invention to provide novel (heterocyclyl)carboxanilide derivatives.

Accordingly, it is an object of the present invention to provide fungicidally active compounds which overcome the disadvantages of the compounds known from the prior art and, in particular, have improved activity at low application rates. Moreover, these compounds should be tolerated well by crop plants and, if possible, cause no or only little damage to useful animals.

We have found that this object is achieved by the (hetero)cyclylcarboxanilides of the formula I described below and their agriculturally acceptable salts,

Accordingly, the present invention relates to (hetero)cyclylcarboxanilides of the formula I, 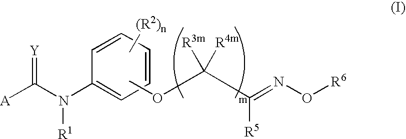
in which variables are as defined below:
A is phenyl or an at least monounsaturated 5- or 6-membered heterocycle having 1, 2 or 3 heteroatoms selected from the group consisting of N, O, S, S(═O) and S(═O)₂ as ring members, where phenyl and the at least monounsaturated 5- or 6-membered heterocycle may be unsubstituted or may carry 1, 2 or 3 radicals R^{a}, where
   R^{a} is halogen, nitro, CN, C₁-C₄-alkyl, C₃-C₆-cycloalkyl, C₂-C₄-alkenyl, C₂-C₄-alkynyl, C₁-C₄-alkoxy, C₁-C₄-haloalkyl, C₃-C₆-halocycloalkyl, C₂-C₄-haloalkenyl, C₂-C₄-haloalkynyl, C₁-C₄-haloalkoxy or phenyl, where phenyl may be unsubstituted or carries one, two or three radicals R^{b} selected from the group consisting of halogen, nitro, CN, C₁-C₄-alkyl, C₃-C₆-cycloalkyl, C₂-C₄-alkenyl, C₂-C₄-alkynyl, C₁-C₄-alkoxy, C₁-C₄-haloalkyl, C₃-C₆-halocycloalkyl, C₂-C₄-haloalkenyl, C₂-C₄-haloalkynyl and C₁-C₄-haloalkoxy;
Y is oxygen or sulfur;
R¹ is H, OH, C₁-C₄-alkyl, C₃-C₆-cycloalkyl, C₁-C₄-alkoxy, C₁-C₄-haloalkyl, C₃-C₆-halocycloalkyl or C₁-C₄-haloalkoxy;
R² is halogen, nitro, CN, C₁-C₄-alkyl, C₃-C₆-cycloalkyl, C₂-C₄-alkenyl, C₂-C₄-alkynyl, C₁-C₄-alkoxy, C₁-C₄-haloalkyl, C₃-C₆-halocycloalkyl, C₂-C₄-haloalkenyl, C₂-C₄-haloalkynyl or C₁-C₄-haloalkoxy;
R^{3m}, R^{4m} are each independently of one another halogen, hydrogen, C₁-C₆-alkyl, C₃-C₆-cycloalkyl, C₂-C₆-alkenyl, C₂-C₆-alkynyl, phenyl, phenyl-C₁-C₄-alkyl, phenyl-C₂-C₄-alkenyl, phenyl-C₂-C₄-alkynyl, C₁-C₆-haloalkyl, C₃-C₆-halocycloalkyl, C₂-C₆-haloalkenyl, C₂-C₆-haloalkynyl, phenyl-C₁-C₄-haloalkyl, phenyl-C₂-C₄-haloalkenyl or phenyl-C₂-C₄-haloalkynyl, where phenyl or the phenyl moiety of phenyl-C₁-C₄-alkyl, phenyl-C₂-C₄-alkenyl, phenyl-C₂-C₄-alkynyl, phenyl-C₁-C₄-haloalkyl, phenyl-C₂-C₄-haloalkenyl and phenyl-C₂-C₄-haloalkynyl may be unsubstituted or may carry one, two or three radicals R^{b}; for m=2 or 3 the variables R³², R⁴² and R³³, R⁴³, respectively, may also be C₁-C₆-alkoxy;
R⁵ is hydrogen, C₁-C₆-alkyl, C₃-C₆-cycloalkyl, C₂-C₆-alkenyl, C₂-C₆-alkynyl, phenyl, phenyl-C₁-C₄-alkyl, phenyl-C₂-C₄-alkenyl, phenyl-C₂-C₄-alkynyl, C₁-C₆-haloalkyl, C₃-C₆-halocycloalkyl, C₂-C₆-haloalkenyl, C₂-C₆-haloalkynyl, phenyl-C₁-C₄-haloalkyl, phenyl-C₂-C₄-haloalkenyl or phenyl-C₂-C₄-haloalkynyl, where phenyl or the phenyl moiety of phenyl-C₁-C₄-alkyl, phenyl-C₂-C₄-alkenyl, phenyl-C₂-C₄-alkynyl, phenyl-C₁-C₄-haloalkyl, phenyl-C₂-C₄-haloalkenyl, phenyl-C₂-C₄-haloalkynyl may be unsubstituted or may carry one, two or three radicals R^{b};
R⁶ is hydrogen, C₁-C₈-alkyl, C₃-C₆-cycloalkyl, C₂-C₈-alkenyl, C₂-C₈-alkynyl, C₁-C₈-haloalkyl, C₃-C₆-halocycloalkyl, C₂-C₈-haloalkenyl, C₂-C₈-haloalkynyl, phenyl, naphthyl, phenyl-C₁-C₆-alkyl, naphthyl-C₁-C₆-alkyl, phenyl-C₂-C₆-alkenyl, phenyl-C₂-C₆-alkynyl, phenyl-C₁-C₆-haloalkyl, phenyl-C₂-C₆-haloalkenyl or phenyl-C₂-C₆-haloalkynyl, where phenyl and naphthyl in the 9 last-mentioned groups may be unsubstituted or may carry 1, 2 or 3 substituents selected from the group consisting of R^{b} and R⁷, where R⁷ is —(CR⁸)═NOR⁹, where
   R⁸ is hydrogen, C₁-C₆-alkyl, C₃-C₆-cycloalkyl, C₂-C₆-alkenyl, C₂-C₆-alkynyl, C₁-C₆-haloalkyl, C₃-C₆-halocycloalkyl, C₂-C₆-haloalkenyl, C₂-C₆-haloalkynyl, phenyl, benzyl; where phenyl and the phenyl group in benzyl may be unsubstituted or may carry one, two or three radicals R^{b}; and
   R⁹ is C₁-C₆-alkyl, C₃-C₆-cycloalkyl, C₂-C₆-alkenyl, C₂-C₆-alkynyl, C₁-C₆-haloalkyl, C₃-C₆-halocycloalkyl, C₂-C₆-haloalkenyl, C₂-C₆-haloalkynyl, phenyl, phenyl-C₁-C₄-alkyl, phenyl-C₁-C₄-haloalkyl, phenyl-C₂-C₄-alkenyl, phenyl-C₂-C₄-haloalkenyl, phenyl-C₂-C₄-alkynyl, phenyl-C₂-C₄-haloalkynyl, where phenyl and the phenyl group in phenyl-C₁-C₄-alkyl, phenyl-C₁-C₄-haloalkyl, phenyl-C₂-C₄-alkenyl, phenyl-C₂-C₄-haloalkenyl, phenyl-C₂-C₄-alkynyl and phenyl-C₂-C₄-haloalkynyl may be unsubstituted or may carry one, two or three radicals R^{b};
n is 0, 1, 2, 3 or 4; and
m is 1, 2 or 3;
   and their agriculturally useful salts.

Moreover, the present invention relates to the use of the (hetero)cyclylcarboxanilides of the formula I and their agriculturally useful salts as fungicides, and to crop protection compositions comprising these compounds.

Furthermore, the present invention relates to a method for controlling phytopathogenic fungi (harmful fungi), which method comprises treating the harmful fungi, their habitat or the plants, areas, materials or spaces to be kept free from them with a fungicidally effective amount of a (hetero)cyclylcarboxanilide of the formula I and/or an agriculturally useful salt of 1.

Depending on the substitution pattern, the compounds of the formula I may contain one or more centers of chirality, in which case they are present as mixtures of enantiomers or diastereomers. The invention provides both the pure enantiomers or diastereomers and their mixtures. Suitable compounds of the formula I also comprise all possible stereoisomers (cis/trans isomers) and mixtures thereof.

Suitable agriculturally useful salts are especially the salts of those cations or the acid addition salts of those acids whose cations or anions, respectively, have no adverse effect on the compounds I. Thus, suitable cations are in particular the ions of the alkali metals, preferably sodium and potassium, of the alkaline earth metals, preferably calcium, magnesium and barium, and of the transition metals, preferably manganese, copper, zinc and iron, and also the ammonium ion which, if desired, may carry one to four C₁-C₄-alkyl substituents and/or one phenyl or benzyl substituent, preferably diisopropylammonium, tetramethylammonium, tetrabutylammonium, trimethylbenzylammonium, furthermore phosphonium ions, sulfonium ions, preferably tri(C₁-C₄-alkyl)sulfonium, and sulfoxonium ions, preferably tri(C₁-C₄-alkyl)sulfoxonium.

Anions of useful acid addition salts are primarily chloride, bromide, fluoride, hydrogensulfate, sulfate, dihydrogenphosphate, hydrogenphosphate, phosphate, nitrate, hydrogencarbonate, carbonate, hexafluorosilicate, hexafluorophosphate, benzoate, and the anions of C₁-C₄-alkanoic acids, preferably formate, acetate, propionate and butyrate. They can be formed by reacting I with an acid of the corresponding anion, preferably hydrochloric acid, hydrobromic acid, sulfuric acid, phosphoric acid, nitric acid.

In the definitions of the variables given in the formulae above, collective terms are used which are generally representative for the substituents in question. The term Cₙ-Cₘ denotes in each case the possible number of carbon atoms in the respective substituent or substituent moiety. All hydrocarbon chains, i.e. all alkyl, haloalkyl, phenylalkyl, alkenyl, haloalkenyl, phenylalkenyl, alkynyl, haloalkynyl and phenylalkynyl moieties can be straight-chain or branched. Halogenated substituents preferably carry one to five identical or different halogen atoms. The term halogen denotes in each case fluorine, chlorine, bromine or iodine.

Examples of other meanings are:
C₁-C₄-alkyl: CH₃, C₂H₅, CH₂—C₂H₅, CH(CH₃)₂, n-butyl, CH(CH₃)—C₂H₅, CH₂—CH(CH₃)₂ or C(CH₃)₃;
C₁-C₄-haloalkyl: a C₁-C₄-alkyl radical as mentioned above which is partially or fully substituted by fluorine, chlorine, bromine and/or iodine, i.e., for example, CH₂F, CHF₂, CF₃, CH₂Cl, CH(Cl)₂, C(Cl)₃, chlorofluoromethyl, dichlorofluoromethyl, chlorodifluoromethyl, 2-fluoroethyl, 2-chloroethyl, 2-bromoethyl, 2-iodoethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, C₂F₅, 2-fluoropropyl, 3-fluoropropyl, 2,2-difluoropropyl, 2,3-difluoropropyl, 2-chloropropyl, 3-chloropropyl, 2,3-dichloropropyl, 2-bromopropyl, 3-bromopropyl, 3,3,3-trifluoropropyl, 3,3,3-trichloropropyl, CH₂—C₂F₅, CF₂—C₂F₅, 1-(fluoromethyl)-2-fluoroethyl, 1-(chloromethyl)-2-chloroethyl, 1-(bromomethyl)-2-bromoethyl, 4-fluorobutyl, 4-chlorobutyl, 4-bromobutyl or nonafluorobutyl;
C₁-C₈-alkyl: a C₁-C₄-alkyl radical as mentioned above, or, for example, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl or 1-ethyl-2-methylpropyl, preferably CH₃, C₂H₅, CH₂—C₂H₅, CH(CH₃)₂, n-butyl, C(CH₃)₃, n-pentyl, n-hexyl, n-heptyl or n-octyl;
C₁-C₈-haloalkyl: a C₁-C₈-alkyl radical as mentioned above which is partially or fully substituted by fluorine, chlorine, bromine and/or iodine, i.e., for example, one of the radicals mentioned under C₁-C₄-haloalkyl or 5-fluoro-1-pentyl, 5-chloro-1-pentyl, 5-bromo-1-pentyl, 5-iodo-1-pentyl, 5,5,5-trichloro-1-pentyl, undecafluoropentyl, 6-fluoro-1-hexyl, 6-chloro-1-hexyl, 6-bromo-1-hexyl, 6-iodo-1-hexyl, 6,6,6-trichloro-1-hexyl or dodecafluorohexyl;
C₂-C₄-alkenyl: unsaturated straight-chain or branched hydrocarbon radicals having 2 to 4 carbon atoms and a double bond in any position, for example ethenyl, 1-propenyl, 2-propenyl, 1-methylethenyl, 1-buten-1-yl, 1-buten-2-yl, 1-buten-3-yl, 2-buten-1-yl, 1-methylprop-1-en-1-yl, 2-methylprop-1-en-1-yl, 1-methylprop-2-en-1-yl, 2-methylprop-2-en-1-yl;
C₂-C₆-alkenyl: C₂-C₄-alkenyl as mentioned above and also, for example: n-penten-1-yl, n-penten-2-yl, n-penten-3-yl, n-penten-4-yl, 1-methylbut-1-en-1-yl, 2-methylbut-1-en-1-yl, 3-methylbut-1-en-1-yl, 1-methylbut-2-en-1-yl, 2-methylbut-2-en-1-yl, 3-methylbut-2-en-1-yl, 1-methylbut-3-en-1-yl, 2-methylbut-3-en-1-yl, 3-methylbut-3-en-1-yl, 1,1-dimethylprop-2-en-1-yl, 1,2-dimethylprop-1-en-1-yl, 1,2-dimethylprop-2-en-1-yl, 1-ethylprop-1-en-2-yl, 1-ethylprop-2-en-1-yl, n-hex-1-en-1-yl, n-hex-2-en-1-yl, n-hex-3-en-1-yl, n-hex-4-en-1-yl, n-hex-5-en-1-yl, 1-methylpent-1-en-1-yl, 2-methylpent-1-en-1-yl, 3-methylpent-1-en-1-yl, 4-methylpent-1-en-1-yl, 1-methylpent-2-en-1-yl, 2-methylpent-2-en-1-yl, 3-methylpent-2-en-1-yl, 4-methylpent-2-en-1-yl, 1-methylpent-3-en-1-yl, 2-methylpent-3-en-1-yl, 3-methylpent-3-en-1-yl, 4-methylpent-3-en-1-yl, 1-methylpent-4-en-1-yl, 2-methylpent-en-1-yl, 3-methylpent-4-en-1-yl, 4-methylpent-4-en-1-yl, 1,1-dimethylbut-2-en-1-yl, 1,1-dimethylbut-3-en-1-yl, 1,2-dimethylbut-1-en-1-yl, 1,2-dimethylbut-2-en-1-yl, 1,2-dimethylbut-3-en-1-yl, 1,3-dimethylbut-3-en-1-yl, 1,3-dimethylbut-2-en-1-yl, 1,3-dimethylbut-3-en-1-yl, 2,2-dimethylbut-3-en-1-yl, 2,3-dimethylbut-1-en-1-yl, 2,3-dimethylbut-2-en-1-yl, 2,3-dimethylbut-3-en-1-yl, 3,3-dimethylbut-1-en-1-yl, 3,3-dimethylbut-2-en-1-yl, 1-ethylbut-1-en-1-yl, 1-ethylbut-2-en-1-yl, 1-ethylbut-3-en-1-yl, 2-ethylbut-1-en-1-yl, 2-ethylbut-2-en-1-yl, 2-ethylbut-3-en-1-yl, 1,1,2-trimethylprop-2-en-1-yl, 1-ethyl-1-methylprop-2-en-1-yl, 1-ethyl-2-methylprop-1-en-1-yl or 1-ethyl-2-methylprop-2-en-1-yl;
C₂-C₄-haloalkenyl: unsaturated straight-chain or branched hydrocarbon radicals having 2 to 4 carbon atoms and a double bond in any position (as mentioned above), where some or all of the hydrogen atoms in these groups are replaced by halogen atoms as mentioned above, in particular by fluorine, chlorine and bromine, i.e., for example, 2-chloroallyl, 3-chloroallyl, 2,3-dichloroallyl, 3,3-dichloroallyl, 2,3,3-trichloroallyl, 2,3-dichlorobut-2-enyl, 2-bromoallyl, 3-bromoallyl, 2,3-dibromoallyl, 3,3-dibromoallyl, 2,3,3-tribromoallyl or 2,3-dibromobut-2-enyl;
C₂-C₆-haloalkenyl: C₂-C₆-alkenyl as mentioned above which is partially or fully substituted by fluorine, chlorine, bromine and/or iodine, for example the radicals mentioned under C₂-C₄-haloalkenyl;
C₂-C₄-alkynyl: straight-chain or branched hydrocarbon groups having 2 to 4 carbon atoms and a triple bond in any position, for example ethynyl, 1-propynyl, 2-propynyl (=propargyl), 1-butynyl, 2-butynyl, 3-butynyl and 1-methyl-2-propynyl;
C₂-C₆-alkynyl: straight-chain or branched hydrocarbon groups having 2 to 6 carbon atoms and a triple bond in any position, for example ethynyl, prop-1-yn-1-yl, prop-2-yn-1-yl, n-but-1-yn-1-yl, n-but-1-yn-3-yl, n-but-1-yn-4-yl, n-but-2-yn-1-yl, n-pent-1-yn-1-yl, n-pent-1-yn-3-yl, n-pent-1-yn-4-yl, n-pent-1-yn-5-yl, n-pent-2-yn-1-yl, n-pent-2-yn-4-yl, n-pent-2-yn-5-yl, 3-methylbut-1-yn-3-yl, 3-methylbut-1-yn-4-yl, n-hex-1-yn-1-yl, n-hex-1-yn-3-yl, n-hex-1-yn-4-yl, n-hex-1-yn-5-yl, n-hex-1-yn-6-yl, n-hex-2-yn-1-yl, n-hex-2-yn-4-yl, n-hex-2-yn-5-yl, n-hex-2-yn-6-yl, n-hex-3-yn-1-yl, n-hex-3-yn-2-yl, 3-methylpent-1-yn-1-yl, 3-methylpent-1-yn-3-yl, 3-methylpent-1-yn-4-yl, 3-methylpent-1-yn-5-yl, 4-methylpent-1-yn-1-yl, 4-methylpent-2-yn-4-yl and 4-methylpent-2-yn-5-yl;
C₂-C₄-haloalkynyl: unsaturated straight-chain or branched hydrocarbon radicals having 2 to 4 carbon atoms and a triple bond in any position (as mentioned above), where some or all of the hydrogen atoms in these groups may be replaced by halogen atoms as mentioned above, in particular by fluorine, chlorine and bromine, i.e., for example, 1,1-difluoroprop-2-yn-1-yl, 4-fluorobut-2-yn-1-yl, 4-chlorobut-2-yn-1-yl or 1,1-difluorobut-2-yn-1-yl,
C₂-C₆-haloalkynyl: C₂-C₆-alkynyl as mentioned above which is partially or fully substituted by fluorine, chlorine, bromine and/or iodine, for example the radicals mentioned under C₂-C₄-haloalkynyl;
C₁-C₄-alkoxy: OCH₃, OC₂H₅, OCH₂—C₂H₅, OCH(CH₃)₂, n-butoxy, OCH(CH₃)—C₂H₅, OCH₂—CH(CH₃)₂ or OC(CH₃)₃;
C₁-C₄-haloalkoxy: a C₁-C₄-alkoxy radical as mentioned above which is partially or fully substituted by fluorine, chlorine, bromine and/or iodine, i.e., for example, OCH₂F, OCHF₂, OCF₃, OCH₂Cl, OCH(Cl)₂, OC(Cl)₃, chlorofluoromethoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, OC₂F₅, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, OCH₂—C₂F₅, OCF₂—C₂F₅, 1-(CH₂F)-2-fluoroethoxy, 1-(CH₂Cl)-2-chloroethoxy, 1-(CH₂Br)-2-bromoethoxy, 4-fluorobutoxy, 4-chlorobutoxy, 4-bromobutoxy or nonafluorobutoxy, preferably OCHF₂, OCF₃, dichlorofluoromethoxy, chlorodifluoromethoxy or 2,2,2-trifluoroethoxy;
C₃-C₆-cycloalkyl: cyclopropyl, cyclobutyl, cyclopentyl or cyclohexyl;
C₃-C₆-cycloalkyl which is unsubstituted or mono- or polysubstituted by halogen: a C₃-C₆-cycloalkyl radical as mentioned above which is unsubstituted or partially or fully substituted by fluorine, chlorine, bromine and/or iodine, i.e., for example, 1-chlorocyclopropyl, 1-fluorocyclopropyl, 2-chlorocyclopropyl, 2-fluorocyclopropyl, 4-chlorocyclohexyl, 4-bromocyclohexyl;
phenyl-C₁-C₄-alkyl: C₁-C₄-alkyl which is substituted by phenyl, for example benzyl, 1- or 2-phenylethyl, 1-, 2- or 3-phenylpropyl, where the phenyl moiety may be unsubstituted or may carry 1, 2 or 3 radicals R^{b}, where R^{b} is selected from the group consisting of halogen, nitro, CN, C₁-C₄-alkyl, C₃-C₆-cycloalkyl, C₂-C₄-alkenyl, C₂-C₄-alkynyl and C₁-C₄-alkoxy, where the 5 last-mentioned groups may be substituted by halogen;
phenyl-C₁-C₄-haloalkyl: C₁-C₄-haloalkyl which is substituted by phenyl, where the phenyl moiety may be unsubstituted or may carry 1, 2 or 3 radicals R^{b}, where R^{b} is selected from the group consisting of halogen, nitro, CN, C₁-C₄-alkyl, C₃-C₆-cycloalkyl, C₂-C₄-alkenyl, C₂-C₄-alkynyl and C₁-C₄-alkoxy, where the 5 last-mentioned groups may be substituted by halogen;
phenyl-C₂-C₄-alkenyl: C₂-C₄-alkenyl which is substituted by phenyl, for example 1- or 2-phenylethenyl, 1-phenylprop-2-en-1-yl, 3-phenyl-1-propen-1-yl, 3-phenyl-2-propen-1-yl, 4-phenyl-1-buten-1-yl or 4-phenyl-2-buten-1-yl; where the phenyl moiety may be unsubstituted or may carry 1, 2 or 3 radicals R^{b}, where R^{b} is selected from the group consisting of halogen, nitro, CN, C₁-C₄-alkyl, C₃-C₆-cycloalkyl, C₂-C₄-alkenyl, C₂-C₄-alkynyl and C₁-C₄-alkoxy, where the 5 last-mentioned groups may be substituted by halogen;
phenyl-C₂-C₄-haloalkenyl: C₂-C₄-haloalkenyl which is substituted by phenyl, where the phenyl moiety may be unsubstituted or may carry 1, 2 or 3 radicals R^{b}, where R^{b} is selected from the group consisting of halogen, nitro, CN, C₁-C₄-alkyl, C₃-C₆-cycloalkyl, C₂-C₄-alkenyl, C₂-C₄-alkynyl and C₁-C₄-alkoxy, where the 5 last-mentioned groups may be substituted by halogen;
phenyl-C₂-C₄-alkynyl: C₂-C₄-alkynyl, which is substituted by phenyl, for example 1-phenyl-2-propyn-1-yl, 3-phenyl-1-propyn-1-yl, 3-phenyl-2-propyn-1-yl, 4-phenyl-1-butyn-1-yl or 4-phenyl-2-butyn-1-yl; where the phenyl moiety of phenyl-C₂-C₄-alkynyl may be unsubstituted or may carry 1, 2 or 3 radicals R^{b}, where R^{b} is selected from the group consisting of halogen, nitro, CN, C₁-C₄-alkyl, C₃-C₆-cycloalkyl, C₂-C₄-alkenyl, C₂-C₄-alkynyl and C₁-C₄-alkoxy, where the 5 last-mentioned groups may be substituted by halogen;
phenyl-C₂-C₄-haloalkynyl: C₂-C₄-haloalkynyl which is substituted by phenyl, where the phenyl moiety may be unsubstituted or may carry 1, 2 or 3 radicals R^{b}, where R^{b} is selected from the group consisting of halogen, nitro, CN, C₁-C₄-alkyl, C₃-C₆-cycloalkyl, C₂-C₄-alkenyl, C₂-C₄-alkynyl and C₁-C₄-alkoxy, where the 5 last-mentioned groups may be substituted by halogen;
an at least monounsaturated heterocycle having 5 or 6 ring members: a monocyclic heterocycle which has one, two or three ring members selected from the group consisting of O, S, S(═O), S(═O)₂ and N and which is at least monounsaturated or fully unsaturated, i.e. aromatic. Examples are 2-furyl and 3-furyl, thienyl, such as 2-thienyl and 3-thienyl, pyrrolyl, such as 2-pyrrolyl and 3-pyrrolyl, isoxazolyl, such as 3-isoxazolyl, 4-isoxazolyl and 5-isoxazolyl, isothiazolyl, such as 3-isothiazolyl, 4-isothiazolyl and 5-isothiazolyl, pyrazolyl, such as 3-pyrazolyl, 4-pyrazolyl and 5-pyrazolyl, oxazolyl, such as 2-oxazolyl, 4-oxazolyl and 5-oxazolyl, thiazolyl, such as 2-thiazolyl, 4-thiazolyl and 5-thiazolyl, imidazolyl, such as 2-imidazolyl and 4-imidazolyl, oxadiazolyl, such as 1,2,4-oxadiazol-3-yl, 1,2,4-oxadiazol-5-yl and 1,3,4-oxadiazol-2-yl, thiadiazolyl, such as 1,2,4-thiadiazol-3-yl, 1,2,4-thiadiazol-5-yl and 1,3,4-thiadiazol-2-yl, triazolyl, such as 1,2,4-triazol-1-yl, 1,2,4-triazol-3-yl and 1,2,4-triazol-4-yl, pyridynyl, such as 2-pyridynyl, 3-pyridynyl and 4-pyridynyl, pyridazynyl, such as 3-pyridazynyl and 4-pyridazynyl, pyrimidynyl, such as 2-pyrimidynyl, 4-pyrimidynyl and 5-pyrimidynyl, 2-pyrazynyl, 1,3,5-triazin-2-yl and 1,2,4-triazin-3-yl, 1,2-dihydrofuran-2-yl, 1,2-dihydrofuran-3-yl, 1,2-dihydrothiophen-2-yl, 1,2-dihydrothiophen-3-yl, 2,3-dihydropyran-4-yl, 2,3-dihydropyran-5-yl, 2,3-dihydropyran-6-yl, 5,6-dihydro-4H-pyran-3-yl, 2,3-dihydrothiopyran-4-yl, 2,3-dihydrothiopyran-5-yl, 2,3-dihydrothiopyran-6-yl, 5,6-dihydro-4H-thiopyran-3-yl, 5,6-dihydro-[1,4]dioxin-2-yl, 5,6-dihydro-[1,4]dithiin-2-yl or 5,6-dihydro[1,4]oxathiin-3-yl, in particular pyridyl, thiazolyl and pyrazolyl.

With a view to the fungicidal activity of the compounds I according to the invention, preference is given to those compounds of the formula I in which A is a cyclic radical A-1 to A-6: 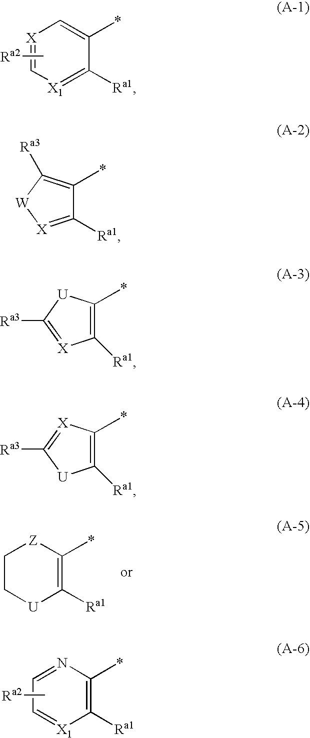
in which * denotes the point of attachment to C(═Y) and the variables are as defined below:
X, X₁ are each independently of one another N or CR^{c}, where R^{c} is H or has one of the meanings mentioned for R_{b}. In particular R^{c} is hydrogen;
W is S or N—R^{a4}, where R^{a4} is hydrogen, C₁-C₄-alkyl, C₁-C₄-haloalkyl, C₁-C₄-alkoxy, C₁-C₄-haloalkyl or phenyl which may be unsubstituted or may carry 1, 2 or 3 radicals R^{b}; R^{a4} in particular is hydrogen, C₁-C₄-alkyl or C₁-C₄-haloalkyl;
U is oxygen or sulfur;
Z is S, S(═O), S(═O)₂ or CH₂, particularly preferably S or CH₂;
R^{a1} is hydrogen, C₁-C₄-alkyl, C₁-C₄-alkoxy, C₁-C₄-haloalkyl, C₁-C₄-haloalkoxy or halogen, particularly preferably hydrogen, halogen, C₁-C₂-alkyl, C₁-C₂-alkoxy, C₁-C₂-fluoroalkoxy or C₁-C₂-fluoroalkyl;
R^{a2} are each independently of one another hydrogen, halogen, nitro, CN, C₁-C₄-alkyl, C₃-C₆-cycloalkyl, C₂-C₄-alkenyl, C₂-C₄-alkynyl, C₁-C₄-alkoxy, where the 5 last-mentioned groups may be substituted by halogen; and
R^{a3} is hydrogen, halogen, nitro, CN, C₁-C₄-alkyl, C₃-C₆-cycloalkyl, C₂-C₄-alkenyl, C₂-C₄-alkynyl, C₁-C₄-alkoxy, where the 5 last-mentioned groups may be substituted by halogen, particularly preferably hydrogen, fluorine, chlorine or C₁-C₄-alkyl.

In the radicals of the formula A-1, A-2, A-3, A-4, A-5 and A-6, the variables R^{a1}, R^{a2} and R^{a3} have in particular the following meanings:
R^{a1} is hydrogen, halogen, in particular fluorine or chlorine, C₁-C₄-alkyl or C₁-C₄-haloalkyl, particularly preferably halogen, trifluoromethyl or methyl;
R^{a2} is hydrogen; and
R^{a3} is halogen, in particular fluorine or chlorine, or methyl.

In the formula A-2, W is preferably a group N—R^{a4}, where R^{a4} is as defined above and has in particular the meanings given as being preferred.

If X in the formulae A-1, A-2, A-3 or A-4 is a group C—R^{c}, R^{c} is preferably hydrogen.

In the formulae A-2, A-3 and A-4, X is in particular N. In the formula A-1, X is in particular CH.

In the formulae A-1 and A-6, X₁ is in particular N.

Examples for radicals A-1 are in particular: 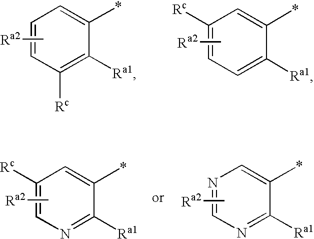
in which *, R^{a1}, R^{a2} and R^{c} have the meanings mentioned above, in particular the preferred meanings.

Examples for radicals A-2 are in particular: 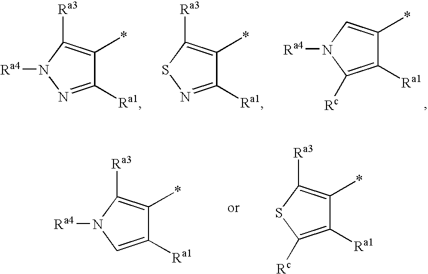
in which *, R^{a1}, R^{a3}, R^{a4} and R^{c} have the meanings mentioned above, in particular the preferred meanings.

Examples for radicals A-3 are in particular: 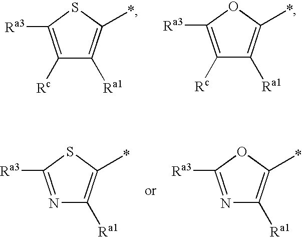
in which *, R^{a1}, R^{a3} and R^{c} have the meanings mentioned above, in particular the preferred meanings.

Examples for radicals A-4 are in particular: 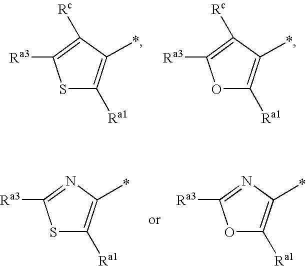
in which *, R^{a1}, R^{a3} and R^{c} have the meanings mentioned above, in particular the preferred meanings.

Examples for radicals A-5 are in particular: 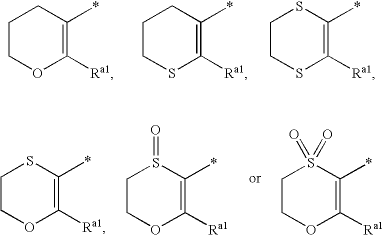
in which * and R^{a1} have the meanings mentioned above, in particular the preferred meanings.

Examples for radicals A-6 are in particular: 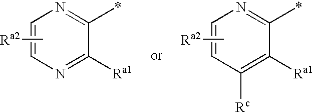
in which *, R^{a1}, R^{a2} and R^{c} have the meanings mentioned above, in particular the preferred meanings.

Examples for radicals A are: 2-chlorophenyl, 2-trifluoromethylphenyl, 2-difluoromethylphenyl, 2-methylphenyl, 2-chloropyridin-3-yl, 2-trifluoromethylpyridin-3-yl, 2-difluoromethylpyridin-3-yl, 2-methylpyridin-3-yl, 4-methylpyrimidin-5-yl, 4-trifluoromethylpyrimidin-5-yl, 4-difluoromethylpyrimidin-5-yl, 1-methyl-3-trifluoromethylpyrazol-4-yl, 1-methyl-3-difluoromethylpyrazol-4-yl, 1,3-dimethylpyrazol-4-yl, 1-methyl-3-trifluoromethyl-5-fluoropyrazol-4-yl, 1-methyl-3-difluoromethyl-5-fluoropyrazol-4-yl, 1-methyl-3-trifluoromethyl-5-chloropyrazol-4-yl, 1-methyl-3-trifluoromethylpyrrol-4-yl, 1-methyl-3-difluoromethylpyrrol-4-yl, 2-methyl-4-trifluoromethylthiazol-5-yl, 2-methyl-4-difluoromethylthiazol-5-yl, 2,4-dimethylthiazol-5-yl, 2-methyl-5-trifluoromethylthiazol-4-yl, 2-methyl-5-difluoromethylthiazol-4-yl, 2,5-dimethylthiazol-4-yl, 2-methyl-4-trifluoromethyloxazol-5-yl, 2-methyl-4-difluoromethyloxazol-5-yl, 2,4-dimethyloxazol-5-yl, 2-trifluoromethylthiophen-3-yl, 5-methyl-2-trifluoromethylthiophen-3-yl, 2-methylthiophen-3-yl, 2,5-dimethylthiophen-3-yl, 3-trifluoromethylthiophen-2-yl, 3-methylthiophen-2-yl, 3,5-dimethylthiophen-2-yl, 5-methyl-3-trifluoromethylthiophen-2-yl, 2-trifluoromethylfuran-3-yl, 5-methyl-2-trifluoromethylfuran-3-yl, 2-methylfuran-3-yl, 2,5-dimethylfuran-3-yl, 2-methyl-5,6-dihydro[1,4]oxathiin-3-yl, 2-methyl-5,6-dihydro-4H-thiopyran-3-yl.

With particular preference, A is a radical A-1a, A-2a or A-3a, 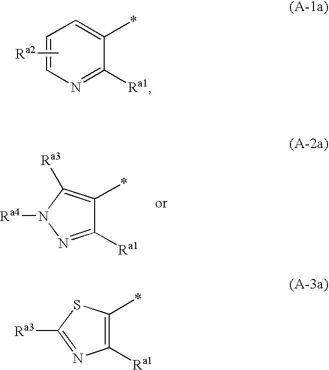
in which *, R^{a1}, R^{a2}, R^{a3} and R^{a4} have the meanings given above, in particular the preferred meanings.

Preference is given to radicals A-1a where R^{a1} is hydrogen, halogen, C₁-C₂-alkyl, C₁-C₂-alkoxy, C₁-C₂-fluoroalkoxy or C₁-C₂-fluoroalkyl; in particular hydrogen, chlorine, bromine, fluorine, methyl, ethyl, methoxy, trifluoromethyl, difluoromethyl, trifluoromethoxy or difluoromethoxy, very particularly preferably fluorine, bromine, chlorine, methyl or trifluoromethyl, and especially chlorine; where R^{a2} is hydrogen, halogen, nitro, CN, C₁-C₄-alkyl, C₃-C₆-cycloalkyl, C₂-C₄-alkenyl, C₂-C₄-alkynyl, C₁-C₄-alkoxy, where the 5 last-mentioned groups may be substituted by halogen, especially hydrogen.

Preference is given to radicals A-2a where: R^{a1} is hydrogen, halogen, C₁-C₂-alkyl, C₁-C₂-alkoxy, C₁-C₂-fluoroalkoxy or C₁-C₂-fluoroalkyl, in particular hydrogen, chlorine, bromine, fluorine, methyl, ethyl, methoxy, trifluoromethyl, difluoromethyl, trifluoromethoxy or difluoromethoxy, very particularly preferably fluorine, bromine, chlorine, methyl or trifluoromethyl, especially trifluoromethyl; R^{a3} is hydrogen, halogen, nitro, CN, C₁-C₄-alkyl, C₃-C₆-cycloalkyl, C₂-C₄-alkenyl, C₂-C₄-alkynyl, C₁-C₄-alkoxy, where the 5 last-mentioned groups may be substituted by halogen, preferably hydrogen, halogen and C₁-C₄-alkyl, in particular halogen, hydrogen; and especially hydrogen; and R^{a4} is hydrogen, C₁-C₄-alkyl, C₁-C₄-haloalkyl, C₁-C₄-alkoxy, C₁-C₄-haloalkyl or phenyl, which may be unsubstituted or may carry 1, 2 or 3 radicals R^{b}, preferably hydrogen, C₁-C₄-alkyl or C₁-C₄-haloalkyl, especially methyl.

Preference is given to radicals A-3a where: R^{a1} is hydrogen, halogen, C₁-C₂-alkyl, C₁-C₂-alkoxy, C₁-C₂-fluoroalkoxy or C₁-C₂-fluoroalkyl, in particular hydrogen, chlorine, bromine, fluorine, methyl, ethyl, methoxy, trifluoromethyl, difluoromethyl, trifluoromethoxy or difluoromethoxy, very particularly preferably fluorine, bromine, chlorine, methyl or trifluoromethyl, especially trifluoromethyl; R^{a3} is hydrogen, halogen, nitro, CN, C₁-C₄-alkyl, C₃-C₆-cycloalkyl, C₂-C₄-alkenyl, C₂-C₄-alkynyl, C₁-C₄-alkoxy, where the 5 last-mentioned groups may be substituted by halogen, preferably hydrogen, halogen or C₁-C₄-alkyl, in particular hydrogen, methyl and especially methyl.

With particular preference, A is selected from the group consisting of:

A-1a where R^{a1}=halogen, especially chlorine and R^{a2}=hydrogen;

A-2a where R^{a1}═C₁-C₂-fluoroalkyl, especially trifluoromethyl, R^{a3}=hydrogen and R^{a4}═C₁-C₄-alkyl, especially methyl; and

A-3a where R^{a1}═C₁-C₂-fluoroalkyl, especially trifluoromethyl, and R^{a3}═C₁-C₄-alkyl, especially methyl.

With a view to their fungicidal activity, preference is given to (hetero)cyclylcarboxanilides of the formula I in which the variables Y, R¹, R², R^{3m}, R^{4m}, R⁵, R⁶, n and m independently of one another and preferably in combination have the following meanings:
Y is O;
R¹ is hydrogen, OH, C₁-C₄-alkyl, in particular H, OH or methyl and especially H;
R² is C₁-C₄-alkyl, C₁-C₄-alkoxy, C₁-C₄-haloalkyl, C₁-C₄-haloalkoxy, nitro, cyano or halogen; particularly preferably C₁-C₄-alkyl, C₁-C₄-alkoxy, nitro, cyano or halogen and especially methyl, methoxy, fluorine, chlorine, bromine, nitro or cyano.
n is 0 or 1, particularly preferably 0;
R^{3m}, R^{4m} are each independently of one another hydrogen, C₁-C₆-alkyl, C₁-C₆-haloalkyl, C₃-C₆-cycloalkyl or C₃-C₆-halocycloalkyl, phenyl, which may be unsubstituted or may carry one, two or three radicals R^{b}; preferably hydrogen or C₁-C₄-alkyl; especially: R³¹ and R⁴¹ are each independently of one another hydrogen, methyl, ethyl;
m is 1;
R⁵ is hydrogen, C₁-C₆-alkyl, C₁-C₆-haloalkyl, C₃-C₆-cycloalkyl, C₃-C₆-halocycloalkyl, phenyl, phenyl-C₁-C₄-alkyl, phenyl-C₁-C₄-haloalkyl, where phenyl in the three last-mentioned radicals may be unsubstituted or may carry one, two or three radicals R^{b}; preferably hydrogen, C₁-C₄-alkyl, C₁-C₄-haloalkyl, phenyl, which may be unsubstituted or may carry one, two or three radicals R^{b};
R⁶ is C₁-C₆-alkyl, C₁-C₆-haloalkyl, C₃-C₆-cycloalkyl, C₃-C₆-halocycloalkyl, C₂-C₆-alkenyl, C₂-C₆-haloalkenyl, C₂-C₄-alkynyl, C₂-C₄-haloalkynyl, phenyl-C₁-C₂-alkyl or phenyl, where phenyl in the two last-mentioned radicals may be unsubstituted or may carry one or two halogen groups, especially fluorine or chlorine.

Particular preference is furthermore given to the (heterocyclyl)carboxanilides of the formula I, in which R¹, R², R^{3m}, R^{4m}, R⁵, R⁶, n and m have the meanings mentioned above and in particular the preferred meanings, Y is oxygen and A is selected from the group consisting of:

A-1, where X and X₁ are each nitrogen, R^{a1} has the meanings mentioned above, in particular the preferred meanings, and is especially methyl, trifluoromethyl, chlorine, bromine or fluorine; R^{a2} has the meanings mentioned above and is especially hydrogen;

A-2, where X is N, W is S, R^{a1} has the meanings mentioned above, in particular the preferred meanings, and is especially methyl, fluorine, chlorine, bromine or trifluoromethyl; R^{a3} has the meanings mentioned above, in particular the preferred meanings, and is especially hydrogen;

A-2, where X is CH, W is N—R^{a4}, where R^{a4} is C₁-C₄-alkyl, especially methyl, R^{a1} has the meanings mentioned above, in particular the preferred meanings and is especially methyl, fluorine, chlorine, bromine or trifluoromethyl; R^{a3} has the meanings mentioned above, in particular the preferred meanings, and is especially hydrogen;

A-3, where U is O, X is N, R^{a1} has the meanings mentioned above, in particular the preferred meanings, and is especially methyl, fluorine, chlorine, bromine or trifluoromethyl; R^{a3} has the meanings mentioned above, in particular the preferred meanings, and is especially hydrogen or methyl;

A-3, where U is S, X is CH, R^{a1} has the meanings mentioned above, in particular the preferred meanings, and is especially methyl, fluorine, chlorine, bromine or trifluoromethyl; R^{a3} has the meanings mentioned above, in particular the preferred meanings, and is especially hydrogen or methyl;

A-4, where U is O, X is CH or N, R^{a1} has the meanings mentioned above, in particular the preferred meanings, and is especially methyl, fluorine, chlorine, bromine or trifluoromethyl; R^{a3} has the meanings mentioned above, in particular the preferred meanings, and is especially hydrogen or methyl;

A-4, where U is S, X is CH or N, R^{a1} has the meanings mentioned above, in particular the preferred meanings, and is especially methyl, fluorine, chlorine, bromine or trifluoromethyl; R^{a3} has the meanings mentioned above, in particular the preferred meanings, and is especially hydrogen or methyl;

A-5, where U is oxygen, Z is CH₂, S, S(═O) or S(═O)₂ and R^{a1} has the meanings mentioned above, in particular the preferred meanings, and is especially methyl, fluorine, chlorine, bromine or trifluoromethyl;

A-6, where X₁ is nitrogen, R^{a2} has the meanings mentioned above and is especially hydrogen; R^{a1} has the meanings mentioned above, in particular the preferred meanings, and is especially methyl, fluorine, chlorine, bromine or trifluoromethyl.

In particular with a view to their use as fungicides and active compounds for controlling pests, preference is given to the individual compounds of the formula Ia (compounds I where R¹═H, n=0) compiled in tables 1 to 42 below, where the variables R⁵, R⁶, R^{3m}, R^{4m} and m each have the meanings given in one row of table A and the variable A has the meaning given in the respective table. In the case of compounds containing double bonds this comprises both the isomerically pure E isomers, Z isomers and isomer mixtures.

TABLE A

| | |
|---|---|
| | (IA) |
| 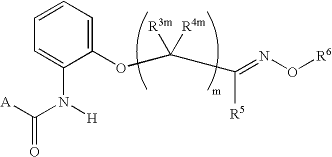 | |

| No. | R⁵ | R⁶ | (C(R^{3m})(R^{4m}))ₘ |
|---|---|---|---|
| 1 | H | CH₃ | —CH₂— |
| 2 | H | C₂H₅ | —CH₂— |
| 3 | H | CH₂CH₂CH₃ | —CH₂— |
| 4 | H | CH(CH₃)₂ | —CH₂— |
| 5 | H | CH₂CH₂CH₂CH₃ | —CH₂— |
| 6 | H | i-C₄H₉ | —CH₂— |
| 7 | H | s-C₄H₉ | —CH₂— |
| 8 | H | C(CH₃)₃ | —CH₂— |
| 9 | H | CH₂CH₂CH₂CH₂CH₃ | —CH₂— |
| 10 | H | CH₂CH₂CH₂CH₂CH₂CH₃ | —CH₂— |
| 11 | H | cyclopentyl | —CH₂— |
| 12 | H | cyclohexyl | —CH₂— |
| 13 | H | allyl | —CH₂— |
| 14 | H | but-2-en-1-yl | —CH₂— |
| 15 | H | 4-chlorobut-2-en-1-yl | —CH₂— |
| 16 | H | propargyl | —CH₂— |
| 17 | H | C₆H₅ | —CH₂— |
| 18 | H | C₆H₅CH₂ | —CH₂— |
| 19 | H | 2-phenyleth-1-yl | —CH₂— |
| 20 | H | 4-Cl—C₆H₄ | —CH₂— |
| 21 | H | 4-F—C₆H₄ | —CH₂— |
| 22 | H | CH₃ | —CH(CH₃)— |
| 23 | H | C₂H₅ | —CH(CH₃)— |
| 24 | H | CH₂CH₂CH₃ | —CH(CH₃)— |
| 25 | H | CH(CH₃)₂ | —CH(CH₃)— |
| 26 | H | CH₂CH₂CH₂CH₃ | —CH(CH₃)— |
| 27 | H | i-C₄H₉ | —CH(CH₃)— |
| 28 | H | s-C₄H₉ | —CH(CH₃)— |
| 29 | H | C(CH₃)₃ | —CH(CH₃)— |
| 30 | H | CH₂CH₂CH₂CH₂CH₃ | —CH(CH₃)— |
| 31 | H | CH₂CH₂CH₂CH₂CH₂CH₃ | —CH(CH₃)— |
| 32 | H | cyclopentyl | —CH(CH₃)— |
| 33 | H | cyclohexyl | —CH(CH₃)— |
| 34 | H | allyl | —CH(CH₃)— |
| 35 | H | but-2-en-1-yl | —CH(CH₃)— |
| 36 | H | 4-chlorobut-2-en-1-yl | —CH(CH₃)— |
| 37 | H | propargyl | —CH(CH₃)— |
| 38 | H | C₆H₅ | —CH(CH₃)— |
| 39 | H | C₆H₅CH₂ | —CH(CH₃)— |
| 40 | H | 2-phenyleth-1-yl | —CH(CH₃)— |
| 41 | H | 4-Cl—C₆H₄ | —CH(CH₃)— |
| 42 | H | 4-F—C₆H₄ | —CH(CH₃)— |
| 43 | H | CH₃ | —CH(C₂H₅)— |
| 44 | H | C₂H₅ | —CH(C₂H₅)— |
| 45 | H | CH₂CH₂CH₃ | —CH(C₂H₅)— |
| 46 | H | CH(CH₃)₂ | —CH(C₂H₅)— |
| 47 | H | CH₂CH₂CH₂CH₃ | —CH(C₂H₅)— |
| 48 | H | i-C₄H₉ | —CH(C₂H₅)— |
| 49 | H | s-C₄H₉ | —CH(C₂H₅)— |
| 50 | H | C(CH₃)₃ | —CH(C₂H₅)— |
| 51 | H | CH₂CH₂CH₂CH₂CH₃ | —CH(C₂H₅)— |
| 52 | H | CH₂CH₂CH₂CH₂CH₂CH₃ | —CH(C₂H₅)— |
| 53 | H | cyclopentyl | —CH(C₂H₅)— |
| 54 | H | cyclohexyl | —CH(C₂H₅)— |
| 55 | H | allyl | —CH(C₂H₅)— |
| 56 | H | but-2-en-1-yl | —CH(C₂H₅)— |
| 57 | H | 4-chlorobut-2-en-1-yl | —CH(C₂H₅)— |
| 58 | H | propargyl | —CH(C₂H₅)— |
| 59 | H | C₆H₅ | —CH(C₂H₅)— |
| 60 | H | C₆H₅CH₂ | —CH(C₂H₅)— |
| 61 | H | 2-phenyleth-1-yl | —CH(C₂H₅)— |
| 62 | H | 4-Cl—C₆H₄ | —CH(C₂H₅)— |
| 63 | H | 4-F—C₆H₄ | —CH(C₂H₅)— |
| 64 | H | CH₃ | —C(CH₃)₂— |
| 65 | H | C₂H₅ | —C(CH₃)₂— |
| 66 | H | CH₂CH₂CH₃ | —C(CH₃)₂— |
| 67 | H | CH(CH₃)₂ | —C(CH₃)₂— |
| 68 | H | CH₂CH₂CH₂CH₃ | —C(CH₃)₂— |
| 69 | H | i-C₄H₉ | —C(CH₃)₂— |
| 70 | H | s-C₄H₉ | —C(CH₃)₂— |
| 71 | H | C(CH₃)₃ | —C(CH₃)₂— |
| 72 | H | CH₂CH₂CH₂CH₂CH₃ | —C(CH₃)₂— |
| 73 | H | CH₂CH₂CH₂CH₂CH₂CH₃ | —C(CH₃)₂— |
| 74 | H | cyclopentyl | —C(CH₃)₂— |
| 75 | H | cyclohexyl | —C(CH₃)₂— |
| 76 | H | allyl | —C(CH₃)₂— |
| 77 | H | but-2-en-1-yl | —C(CH₃)₂— |
| 78 | H | 4-chlorobut-2-en-1-yl | —C(CH₃)₂— |
| 79 | H | propargyl | —C(CH₃)₂— |
| 80 | H | C₆H₅ | —C(CH₃)₂— |
| 81 | H | C₆H₅CH₂ | —C(CH₃)₂— |
| 82 | H | 2-phenyleth-1-yl | —C(CH₃)₂— |
| 83 | H | 4-Cl—C₆H₄ | —C(CH₃)₂— |
| 84 | H | 4-F—C₆H₄ | —C(CH₃)₂— |
| 85 | H | CH₃ | —CH₂CH₂— |
| 86 | H | C₂H₅ | —CH₂CH₂— |
| 87 | H | CH₂CH₂CH₃ | —CH₂CH₂— |
| 88 | H | CH(CH₃)₂ | —CH₂CH₂— |
| 89 | H | CH₂CH₂CH₂CH₃ | —CH₂CH₂— |
| 90 | H | i-C₄H₉ | —CH₂CH₂— |
| 91 | H | s-C₄H₉ | —CH₂CH₂— |
| 92 | H | C(CH₃)₃ | —CH₂CH₂— |
| 93 | H | CH₂CH₂CH₂CH₂CH₃ | —CH₂CH₂— |
| 94 | H | CH₂CH₂CH₂CH₂CH₂CH₃ | —CH₂CH₂— |
| 95 | H | cyclopentyl | —CH₂CH₂— |
| 96 | H | cyclohexyl | —CH₂CH₂— |
| 97 | H | allyl | —CH₂CH₂— |
| 98 | H | but-2-en-1-yl | —CH₂CH₂— |
| 99 | H | 4-chlorobut-2-en-1-yl | —CH₂CH₂— |
| 100 | H | propargyl | —CH₂CH₂— |
| 101 | H | C₆H₅ | —CH₂CH₂— |
| 102 | H | C₆H₅CH₂ | —CH₂CH₂— |
| 103 | H | 2-phenyleth-1-yl | —CH₂CH₂— |
| 104 | H | 4-Cl—C₆H₄ | —CH₂CH₂— |
| 105 | H | 4-F—C₆H₄ | —CH₂CH₂— |
| 106 | H | CH₃ | —CH(CH₃)CH₂— |
| 107 | H | C₂H₅ | —CH(CH₃)CH₂— |
| 108 | H | CH₂CH₂CH₃ | —CH(CH₃)CH₂— |
| 109 | H | CH(CH₃)₂ | —CH(CH₃)CH₂— |
| 110 | H | CH₂CH₂CH₂CH₃ | —CH(CH₃)CH₂— |
| 111 | H | i-C₄H₉ | —CH(CH₃)CH₂— |
| 112 | H | s-C₄H₉ | —CH(CH₃)CH₂— |
| 113 | H | C(CH₃)₃ | —CH(CH₃)CH₂— |
| 114 | H | CH₂CH₂CH₂CH₂CH₃ | —CH(CH₃)CH₂— |
| 115 | H | CH₂CH₂CH₂CH₂CH₂CH₃ | —CH(CH₃)CH₂— |
| 116 | H | cyclopentyl | —CH(CH₃)CH₂— |
| 117 | H | cyclohexyl | —CH(CH₃)CH₂— |
| 118 | H | allyl | —CH(CH₃)CH₂— |
| 119 | H | but-2-en-1-yl | —CH(CH₃)CH₂— |
| 120 | H | 4-chlorobut-2-en-1-yl | —CH(CH₃)CH₂— |
| 121 | H | propargyl | —CH(CH₃)CH₂— |
| 122 | H | C₆H₅ | —CH(CH₃)CH₂— |
| 123 | H | C₆H₅CH₂ | —CH(CH₃)CH₂— |
| 124 | H | 2-phenyleth-1-yl | —CH(CH₃)CH₂— |
| 125 | H | 4-Cl—C₈H₄ | —CH(CH₃)CH₂— |
| 126 | H | 4-F—C₆H₄ | —CH(CH₃)CH₂— |
| 127 | H | CH₃ | —CH₂CH(CH₃)— |
| 128 | H | C₂H₅ | —CH₂CH(CH₃)— |
| 129 | H | CH₂CH₂CH₃ | —CH₂CH(CH₃)— |
| 130 | H | CH(CH₃)₂ | —CH₂CH(CH₃)— |
| 131 | H | CH₂CH₂CH₂CH₃ | —CH₂CH(CH₃)— |
| 132 | H | i-C₄H₉ | —CH₂CH(CH₃)— |
| 133 | H | s-C₄H₉ | —CH₂CH(CH₃)— |
| 134 | H | C(CH₃)₃ | —CH₂CH(CH₃)— |
| 135 | H | CH₂CH₂CH₂CH₂CH₃ | —CH₂CH(CH₃)— |
| 136 | H | CH₂CH₂CH₂CH₂CH₂CH₃ | —CH₂CH(CH₃)— |
| 137 | H | cyclopentyl | —CH₂CH(CH₃)— |
| 138 | H | cyclohexyl | —CH₂CH(CH₃)— |
| 139 | H | allyl | —CH₂CH(CH₃)— |
| 140 | H | but-2-en-1-yl | —CH₂CH(CH₃)— |
| 141 | H | 4-chlorobut-2-en-1-yl | —CH₂CH(CH₃)— |
| 142 | H | propargyl | —CH₂CH(CH₃)— |
| 143 | H | C₆H₅ | —CH₂CH(CH₃)— |
| 144 | H | C₆H₅CH₂ | —CH₂CH(CH₃)— |
| 145 | H | 2-phenyleth-1-yl | —CH₂CH(CH₃)— |
| 146 | H | 4-Cl—C₆H₄ | —CH₂CH(CH₃)— |
| 147 | H | 4-F—C₆H₄ | —CH₂CH(CH₃)— |
| 148 | H | CH₃ | —CH(CH₃)CH(CH₃)— |
| 149 | H | C₂H₅ | —CH(CH₃)CH(CH₃)— |
| 150 | H | CH₂CH₂C_{H3} | —CH(CH₃)CH(CH₃)— |
| 151 | H | CH(CH₃)₂ | —CH(CH₃)CH(CH₃)— |
| 152 | H | CH₂CH₂CH₂CH₃ | —CH(CH₃)CH(CH₃)— |
| 153 | H | i-C₄H₉ | —CH(CH₃)CH(CH₃)— |
| 154 | H | s-C₄H₉ | —CH(CH₃)CH(CH₃)— |
| 155 | H | C(CH₃)₃ | —CH(CH₃)CH(CH₃)— |
| 156 | H | CH₂CH₂CH₂CH₂CH₃ | —CH(CH₃)CH(CH₃)— |
| 157 | H | CH₂CH₂CH₂CH₂CH₂CH₃ | —CH(CH₃)CH(CH₃)— |
| 158 | H | cyclopentyl | —CH(CH₃)CH(CH₃)— |
| 159 | H | cyclohexyl | —CH(CH₃)CH(CH₃)— |
| 160 | H | allyl | —CH(CH₃)CH(CH₃)— |
| 161 | H | but-2-en-1-yl | —CH(CH₃)CH(CH₃)— |
| 162 | H | 4-chlorobut-2-en-1-yl | —CH(CH₃)CH(CH₃)— |
| 163 | H | propargyl | —CH(CH₃)CH(CH₃)— |
| 164 | H | C₆H₅ | —CH(CH₃)CH(CH₃)— |
| 165 | H | C₆H₅CH₂ | —CH(CH₃)CH(CH₃)— |
| 166 | H | 2-phenyleth-1-yl | —CH(CH₃)CH(CH₃)— |
| 167 | H | 4-Cl—C₆H₄ | —CH(CH₃)CH(CH₃)— |
| 168 | H | 4-F—C₆H₄ | —CH(CH₃)CH(CH₃)— |
| 169 | H | CH₃ | —CH₂CH₂CH₂— |
| 170 | H | C₂H₅ | —CH₂CH₂CH₂— |
| 171 | H | CH₂CH₂CH₃ | —CH₂CH₂CH₂— |
| 172 | H | CH(CH₃)₂ | —CH₂CH₂CH₂— |
| 173 | H | CH₂CH₂CH₂CH₃ | —CH₂CH₂CH₂— |
| 174 | H | i-C₄H₉ | —CH₂CH₂CH₂— |
| 175 | H | s-C₄H₉ | —CH₂CH₂CH₂— |
| 176 | H | C(CH₃)₃ | —CH₂CH₂CH₂— |
| 177 | H | CH₂CH₂CH₂CH₂CH₃ | —CH₂CH₂CH₂— |
| 178 | H | CH₂CH₂CH₂CH₂CH₂CH₃ | —CH₂CH₂CH₂— |
| 179 | H | cyclopentyl | —CH₂CH₂CH₂— |
| 180 | H | cyclohexyl | —CH₂CH₂CH₂— |
| 181 | H | allyl | —CH₂CH₂CH₂— |
| 182 | H | but-2-en-1-yl | —CH₂CH₂CH₂— |
| 183 | H | 4-chlorobut-2-en-1-yl | —CH₂CH₂CH₂— |
| 184 | H | propargyl | —CH₂CH₂CH₂— |
| 185 | H | C₆H₅ | —CH₂CH₂CH₂— |
| 186 | H | C₆H₅CH₂ | —CH₂CH₂CH₂— |
| 187 | H | 2-phenyleth-1-yl | —CH₂CH₂CH₂— |
| 188 | H | 4-Cl—C₆H₄ | —CH₂CH₂CH₂— |
| 189 | H | 4-F—C₆H₄ | —CH₂CH₂CH₂— |
| 190 | CH₃ | CH₃ | —CH₂— |
| 191 | CH₃ | C₂H₅ | —CH₂— |
| 192 | CH₃ | CH₂CH₂CH₃ | —CH₂— |
| 193 | CH₃ | CH(CH₃)₂ | —CH₂— |
| 194 | CH₃ | CH₂CH₂CH₂CH₃ | —CH₂— |
| 195 | CH₃ | i-C₄H₉ | —CH₂— |
| 196 | CH₃ | s-C₄H₉ | —CH₂— |
| 197 | CH₃ | C(CH₃)₃ | —CH₂— |
| 198 | CH₃ | CH₂CH₂CH₂CH₂CH₃ | —CH₂— |
| 199 | CH₃ | CH₂CH₂CH₂CH₂CH₂CH₃ | —CH₂— |
| 200 | CH₃ | cyclopentyl | —CH₂— |
| 201 | CH₃ | cyclohexyl | —CH₂— |
| 202 | CH₃ | allyl | —CH₂— |
| 203 | CH₃ | but-2-en-1-yl | —CH₂— |
| 204 | CH₃ | 4-chlorobut-2-en-1-yl | —CH₂— |
| 205 | CH₃ | propargyl | —CH₂— |
| 206 | CH₃ | C₆H₅ | —CH₂— |
| 207 | CH₃ | C₆H₅CH₂ | —CH₂— |
| 208 | CH₃ | 2-phenyleth-1-yl | —CH₂— |
| 209 | CH₃ | 4-Cl—C₆H₄ | —CH₂— |
| 210 | CH₃ | 4-F—C₆H₄ | —CH₂— |
| 211 | CH₃ | CH₃ | —CH(CH₃)— |
| 212 | CH₃ | C₂H₅ | —CH(CH₃)— |
| 213 | CH₃ | CH₂CH₂CH₃ | —CH(CH₃)— |
| 214 | CH₃ | CH(CH₃)₂ | —CH(CH₃)— |
| 215 | CH₃ | CH₂CH₂CH₂CH₃ | —CH(CH₃)— |
| 216 | CH₃ | i-C₄H₉ | —CH(CH₃)— |
| 217 | CH₃ | s-C₄H₉ | —CH(CH₃)— |
| 218 | CH₃ | C(CH₃)₃ | —CH(CH₃)— |
| 219 | CH₃ | CH₂CH₂CH₂CH₂CH₃ | —CH(CH₃)— |
| 220 | CH₃ | CH₂CH₂CH₂CH₂CH₂CH₃ | —CH(CH₃)— |
| 221 | CH₃ | cyclopentyl | —CH(CH₃)— |
| 222 | CH₃ | cyctohexyl | —CH(CH₃)— |
| 223 | CH₃ | allyl | —CH(CH₃)— |
| 224 | CH₃ | but-2-en-1-yl | —CH(CH₃)— |
| 225 | CH₃ | 4-chlorobut-2-en-1-yl | —CH(CH₃)— |
| 226 | CH₃ | propargyl | —CH(CH₃)— |
| 227 | CH₃ | C₆H₅ | —CH(CH₃)— |
| 228 | CH₃ | C₆H₅CH₂ | —CH(CH₃)— |
| 229 | CH₃ | 2-phenyleth-1-yl | —CH(CH₃)— |
| 230 | CH₃ | 4-Cl—C₆H₄ | —CH(CH₃)— |
| 231 | CH₃ | 4-F—C₆H₄ | —CH(CH₃)— |
| 232 | CH₃ | CH₃ | —CH(C₂H₅)— |
| 233 | CH₃ | C₂H₅ | —CH(C₂H₅)— |
| 234 | CH₃ | CH₂CH₂CH₃ | —CH(C₂H₅)— |
| 235 | CH₃ | CH(CH₃)₂ | —CH(C₂H₅)— |
| 236 | CH₃ | CH₂CH₂CH₂CH₃ | —CH(C₂H₅)— |
| 237 | CH₃ | i-C₄H₉ | —CH(C₂H₅)— |
| 238 | CH₃ | s-C₄H₉ | —CH(C₂H₅)— |
| 239 | CH₃ | C(CH₃)₃ | —CH(C₂H₅)— |
| 240 | CH₃ | CH₂CH₂CH₂CH₂CH₃ | —CH(C₂H₅)— |
| 241 | CH₃ | CH₂CH₂CH₂CH₂CH₂CH₃ | —CH(C₂H₅)— |
| 242 | CH₃ | cyclopentyl | —CH(C₂H₅)— |
| 243 | CH₃ | cyclohexyl | —CH(C₂H₅)— |
| 244 | CH₃ | allyl | —CH(C₂H₅)— |
| 245 | CH₃ | but-2-en-1-yl | —CH(C₂H₅)— |
| 246 | CH₃ | 4-chlorobut-2-en-1-yl | —CH(C₂H₅)— |
| 247 | CH₃ | propargyl | —CH(C₂H₅)— |
| 248 | CH₃ | C₆H₅ | —CH(C₂H₅)— |
| 249 | CH₃ | C₆H₅CH₂ | —CH(C₂H₅)— |
| 250 | CH₃ | 2-phenyleth-1-yl | —CH(C₂H₅)— |
| 251 | CH₃ | 4-Cl—C₆H₄ | —CH(C₂H₅)— |
| 252 | CH₃ | 4-F—C₆H₄ | —CH(C₂H₅)— |
| 253 | CH₃ | CH₃ | —C(CH₃)₂— |
| 254 | CH₃ | C₂H₅ | —C(CH₃)₂— |
| 255 | CH₃ | CH₂CH₂CH₃ | —C(CH₃)₂— |
| 256 | CH₃ | CH(CH₃)₂ | —C(CH₃)₂— |
| 257 | CH₃ | CH₂CH₂CH₂CH₃ | —C(CH₃)₂— |
| 258 | CH₃ | i-C₄H₉ | —C(CH₃)₂— |
| 259 | CH₃ | s-C₄H₉ | —C(CH₃)₂— |
| 260 | CH₃ | C(CH₃)₃ | —C(CH₃)₂— |
| 261 | CH₃ | CH₂CH₂CH₂CH₂CH₃ | —C(CH₃)₂— |
| 262 | CH₃ | CH₂CH₂CH₂CH₂CH₂CH₃ | —C(CH₃)₂— |
| 263 | CH₃ | cyclopentyl | —C(CH₃)₂— |
| 264 | CH₃ | cyclohexyl | —C(CH₃)₂— |
| 265 | CH₃ | allyl | —C(CH₃)₂— |
| 266 | CH₃ | but-2-en-1-yl | —C(CH₃)₂— |
| 267 | CH₃ | 4-chlorobut-2-en-1-yl | —C(CH₃)₂— |
| 268 | CH₃ | propargyl | —C(CH₃)₂— |
| 269 | CH₃ | C₆H₅ | —C(CH₃)₂— |
| 270 | CH₃ | C₆H₅CH₂ | —C(CH₃)₂— |
| 271 | CH₃ | 2-phenyleth-1-yl | —C(CH₃)₂— |
| 272 | CH₃ | 4-Cl—C₆H₄ | —C(CH₃)₂— |
| 273 | CH₃ | 4-F—C₆H₄ | —C(CH₃)₂— |
| 274 | CH₃ | CH₃ | —CH₂CH₂— |
| 275 | CH₃ | C₂H₅ | —CH₂CH₂— |
| 276 | CH₃ | CH₂CH₂CH₃ | —CH₂CH₂— |
| 277 | CH₃ | CH(CH₃)₂ | —CH₂CH₂— |
| 278 | CH₃ | CH₂CH₂CH₂CH₃ | —CH₂CH₂— |
| 279 | CH₃ | i-C₄H₉ | —CH₂CH₂— |
| 280 | CH₃ | s-C₄H₉ | —CH₂CH₂— |
| 281 | CH₃ | C(CH₃)₃ | —CH₂CH₂— |
| 282 | CH₃ | CH₂CH₂CH₂CH₂CH₃ | —CH₂CH₂— |
| 283 | CH₃ | CH₂CH₂CH₂CH₂CH₂CH₃ | —CH₂CH₂— |
| 284 | CH₃ | cyclopentyl | —CH₂CH₂— |
| 285 | CH₃ | cyclohexyl | —CH₂CH₂— |
| 286 | CH₃ | allyl | —CH₂CH₂— |
| 287 | CH₃ | but-2-en-1-yl | —CH₂CH₂— |
| 288 | CH₃ | 4-chlorobut-2-en-1-yl | —CH₂CH₂— |
| 289 | CH₃ | propargyl | —CH₂CH₂— |
| 290 | CH₃ | C₆H₅ | —CH₂CH₂— |
| 291 | CH₃ | C₆H₅CH₂ | —CH₂CH₂— |
| 292 | CH₃ | 2-phenyleth-1-yl | —CH₂CH₂— |
| 293 | CH₃ | 4-Cl—C₆H₄ | —CH₂CH₂— |
| 294 | CH₃ | 4-F—C₆H₄ | —CH₂CH₂— |
| 295 | CH₃ | CH₃ | —CH(CH₃)CH₂— |
| 296 | CH₃ | C₂H₅ | —CH(CH₃)CH₂— |
| 297 | CH₃ | CH₂CH₂CH₃ | —CH(CH₃)CH₂— |
| 298 | CH₃ | CH(CH₃)₂ | —CH(CH₃)CH₂— |
| 299 | CH₃ | CH₂CH₂CH₂CH₃ | —CH(CH₃)CH₂— |
| 300 | CH₃ | i-C₄H₉ | —CH(CH₃)CH₂— |
| 301 | CH₃ | s-C₄H₉ | —CH(CH₃)CH₂— |
| 302 | CH₃ | C(CH₃)₃ | —CH(CH₃)CH₂— |
| 303 | CH₃ | CH₂CH₂CH₂CH₂CH₃ | —CH(CH₃)CH₂— |
| 304 | CH₃ | CH₂CH₂CH₂CH₂CH₂CH₃ | —CH(CH₃)CH₂— |
| 305 | CH₃ | cyclopentyl | —CH(CH₃)CH₂— |
| 306 | CH₃ | cyclohexyl | —CH(CH₃)CH₂— |
| 307 | CH₃ | allyl | —CH(CH₃)CH₂— |
| 308 | CH₃ | but-2-en-1-yl | —CH(CH₃)CH₂— |
| 309 | CH₃ | 4-chlorobut-2-en-1-yl | —CH(CH₃)CH₂— |
| 310 | CH₃ | propargyl | —CH(CH₃)CH₂— |
| 311 | CH₃ | C₆H₅ | —CH(CH₃)CH₂— |
| 312 | CH₃ | C₆H₅CH₂ | —CH(CH₃)CH₂— |
| 313 | CH₃ | 2-phenyleth-1-yl | —CH(CH₃)CH₂— |
| 314 | CH₃ | 4-Cl—C₆H₄ | —CH(CH₃)CH₂— |
| 315 | CH₃ | 4-F—C₆H₄ | —CH(CH₃)CH₂— |
| 316 | CH₃ | CH₃ | —CH₂CH(CH₃)— |
| 317 | CH₃ | C₂H₅ | —CH₂CH(CH₃)— |
| 318 | CH₃ | CH₂CH₂CH₃ | —CH₂CH(CH₃)— |
| 319 | CH₃ | CH(CH₃)₂ | —CH₂CH(CH₃)— |
| 320 | CH₃ | CH₂CH₂CH₂CH₃ | —CH₂CH(CH₃)— |
| 321 | CH₃ | i-C₄H₉ | —CH₂CH(CH₃)— |
| 322 | CH₃ | s-C₄H₉ | —CH₂CH(CH₃)— |
| 323 | CH₃ | C(CH₃)₃ | —CH₂CH(CH₃)— |
| 324 | CH₃ | CH₂CH₂CH₂CH₂CH₃ | —CH₂CH(CH₃)— |
| 325 | CH₃ | CH₂CH₂CH₂CH₂CH₂CH₃ | —CH₂CH(CH₃)— |
| 326 | CH₃ | cyclopentyl | —CH₂CH(CH₃)— |
| 327 | CH₃ | cyclohexyl | —CH₂CH(CH₃)— |
| 328 | CH₃ | allyl | —CH₂CH(CH₃)— |
| 329 | CH₃ | but-2-en-1-yl | —CH₂CH(CH₃)— |
| 330 | CH₃ | 4-chlorobut-2-en-1-yl | —CH₂CH(CH₃)— |
| 331 | CH₃ | propargyl | —CH₂CH(CH₃)— |
| 332 | CH₃ | C₆H₅ | —CH₂CH(CH₃)— |
| 333 | CH₃ | C₆H₅CH₂ | —CH₂CH(CH₃)— |
| 334 | CH₃ | 2-phenyleth-1-yl | —CH₂CH(CH₃)— |
| 335 | CH₃ | 4-Cl—C₆H₄ | —CH₂CH(CH₃)— |
| 336 | CH₃ | 4-F—C₆H₄ | —CH₂CH(CH₃)— |
| 337 | CH₃ | CH₃ | —CH(CH₃)CH(CH₃)— |
| 338 | CH₃ | C₂H₅ | —CH(CH₃)CH(CH₃)— |
| 339 | CH₃ | CH₂CH₂CH₃ | —CH(CH₃)CH(CH₃)— |
| 340 | CH₃ | CH(CH₃)₂ | —CH(CH₃)CH(CH₃)— |
| 341 | CH₃ | CH₂CH₂CH₂CH₃ | —CH(CH₃)CH(CH₃)— |
| 342 | CH₃ | i-C₄H₉ | —CH(CH₃)CH(CH₃)— |
| 343 | CH₃ | s-C₄H₉ | —CH(CH₃)CH(CH₃)— |
| 344 | CH₃ | C(CH₃)₃ | —CH(CH₃)CH(CH₃)— |
| 345 | CH₃ | CH₂CH₂CH₂CH₂CH₃ | —CH(CH₃)CH(CH₃)— |
| 346 | CH₃ | CH₂CH₂CH₂CH₂CH₂CH₃ | —CH(CH₃)CH(CH₃)— |
| 347 | CH₃ | cyclopentyl | —CH(CH₃)CH(CH₃)— |
| 348 | CH₃ | cyclohexyl | —CH(CH₃)CH(CH₃)— |
| 349 | CH₃ | allyl | —CH(CH₃)CH(CH₃)— |
| 350 | CH₃ | but-2-en-1-yl | —CH(CH₃)CH(CH₃)— |
| 351 | CH₃ | 4-chlorobut-2-en-1-yl | —CH(CH₃)CH(CH₃)— |
| 352 | CH₃ | propargyl | —CH(CH₃)CH(CH₃)— |
| 353 | CH₃ | C₆H₅ | —CH(CH₃)CH(CH₃)— |
| 354 | CH₃ | C₆H₅CH₂ | —CH(CH₃)CH(CH₃)— |
| 355 | CH₃ | 2-phenyleth-1-yl | —CH(CH₃)CH(CH₃)— |
| 356 | CH₃ | 4-Cl—C₆H₄ | —CH(CH₃)CH(CH₃)— |
| 357 | CH₃ | 4-F—C₆H₄ | —CH(CH₃)CH(CH₃)— |
| 358 | CH₃ | CH₃ | —CH₂CH₂CH₂— |
| 359 | CH₃ | C₂H₅ | —CH₂CH₂CH₂— |
| 360 | CH₃ | CH₂CH₂CH₃ | —CH₂CH₂CH₂— |
| 361 | CH₃ | CH(CH₃)₂ | —CH₂CH₂CH₂— |
| 362 | CH₃ | CH₂CH₂CH₂CH₃ | —CH₂CH₂CH₂— |
| 363 | CH₃ | i-C₄H₉ | —CH₂CH₂CH₂— |
| 364 | CH₃ | s-C₄H₉ | —CH₂CH₂CH₂— |
| 365 | CH₃ | C(CH₃)₃ | —CH₂CH₂CH₂— |
| 366 | CH₃ | CH₂CH₂CH₂CH₂CH₃ | —CH₂CH₂CH₂— |
| 367 | CH₃ | CH₂CH₂CH₂CH₂CH₂CH₃ | —CH₂CH₂CH₂— |
| 368 | CH₃ | cyclopentyl | —CH₂CH₂CH₂— |
| 369 | CH₃ | cyclohexyl | —CH₂CH₂CH₂— |
| 370 | CH₃ | ailyl | —CH₂CH₂CH₂— |
| 371 | CH₃ | but-2-en-1-yl | —CH₂CH₂CH₂— |
| 372 | CH₃ | 4-chlorobut-2-en-1-yl | —CH₂CH₂CH₂— |
| 373 | CH₃ | propargyl | —CH₂CH₂CH₂— |
| 374 | CH₃ | C₆H₅ | —CH₂CH₂CH₂— |
| 375 | CH₃ | C₆H₅CH₂ | —CH₂CH₂CH₂— |
| 376 | CH₃ | 2-phenyleth-1-yl | —CH₂CH₂CH₂— |
| 377 | CH₃ | 4-Cl—C₆H₄ | —CH₂CH₂CH₂— |
| 378 | CH₃ | 4-F—C₆H₄ | —CH₂CH₂CH₂— |
| 379 | C₂H₅ | CH₃ | —CH₂— |
| 380 | C₂H₅ | C₂H₅ | —CH₂— |
| 381 | C₂H₅ | CH₂CH₂CH₃ | —CH₂— |
| 382 | C₂H₅ | CH(CH₃)₂ | —CH₂— |
| 383 | C₂H₅ | CH₂CH₂CH₂CH₃ | —CH₂— |
| 384 | C₂H₅ | i-C₄H₉ | —CH₂— |
| 385 | C₂H₅ | s-C₄H₉ | —CH₂— |
| 386 | C₂H₅ | C(CH₃)₃ | —CH₂— |
| 387 | C₂H₅ | CH₂CH₂CH₂CH₂CH₃ | —CH₂— |
| 388 | C₂H₅ | CH₂CH₂CH₂CH₂CH₂CH₃ | —CH₂— |
| 389 | C₂H₅ | cyclopentyl | —CH₂— |
| 390 | C₂H₅ | cyclohexyl | —CH₂— |
| 391 | C₂H₅ | allyl | —CH₂— |
| 392 | C₂H₅ | but-2-en-1-yl | —CH₂— |
| 393 | C₂H₅ | 4-chlorobut-2-en-1-yl | —CH₂— |
| 394 | C₂H₅ | propargyl | —CH₂— |
| 395 | C₂H₅ | C₆H₅ | —CH₂— |
| 396 | C₂H₅ | C₆H₅CH₂ | —CH₂— |
| 397 | C₂H₅ | 2-phenyleth-1-yl | —CH₂— |
| 398 | C₂H₅ | 4-Cl—C₆H₄ | —CH₂— |
| 399 | C₂H₅ | 4-F—C₆H₄ | —CH₂— |
| 400 | C₂H₅ | CH₃ | —CH(CH₃)— |
| 401 | C₂H₅ | C₂H₅ | —CH(CH₃)— |
| 402 | C₂H₅ | CH₂CH₂CH₃ | —CH(CH₃)— |
| 403 | C₂H₅ | CH(CH₃)₂ | —CH(CH₃)— |
| 404 | C₂H₅ | CH₂CH₂CH₂CH₃ | —CH(CH₃)— |
| 405 | C₂H₅ | i-C₄H₉ | —CH(CH₃)— |
| 406 | C₂H₅ | s-C₄H₉ | —CH(CH₃)— |
| 407 | C₂H₅ | C(CH₃)₃ | —CH(CH₃)— |
| 408 | C₂H₅ | CH₂CH2CH₂CH₂CH₃ | —CH(CH₃)— |
| 409 | C₂H₅ | CH₂CH2CH₂CH₂CH₂CH₃ | —CH(CH₃)— |
| 410 | C₂H₅ | cyclopentyl | —CH(CH₃)— |
| 411 | C₂H₅ | cyclohexyl | —CH(CH₃)— |
| 412 | C₂H₅ | allyl | —CH(CH₃)— |
| 413 | C₂H₅ | but-2-en-1-yl | —CH(CH₃)— |
| 414 | C₂H₅ | 4-chlorobut-2-en-1-yl | —CH(CH₃)— |
| 415 | C₂H₅ | propargyl | —CH(CH₃)— |
| 416 | C₂H₅ | C₆H₅ | —CH(CH₃)— |
| 417 | C₂H₅ | C₆H₅CH₂ | —CH(CH₃)— |
| 418 | C₂H₅ | 2-phenyleth-1-yl | —CH(CH₃)— |
| 419 | C₂H₅ | 4-Cl—C₆H₄ | —CH(CH₃)— |
| 420 | C₂H₅ | 4-F—C₆H₄ | —CH(CH₃)— |
| 421 | C₂H₅ | CH₃ | —CH(C₂H₅)— |
| 422 | C₂H₅ | C₂H₅ | —CH(C₂H₅)— |
| 423 | C₂H₅ | CH₂CH₂CH₃ | —CH(C₂H₅)— |
| 424 | C₂H₅ | CH(CH₃)₂ | —CH(C₂H₅)— |
| 425 | C₂H₅ | CH₂CH₂CH₂CH₃ | —CH(C₂H₅)— |
| 426 | C₂H₅ | i-C₄H₉ | —CH(C₂H₅)— |
| 427 | C₂H₅ | s-C₄H₉ | —CH(C₂H₅)— |
| 428 | C₂H₅ | C(CH₃)₃ | —CH(C₂H₅)— |
| 429 | C₂H₅ | CH₂CH₂CH₂CH₂CH₃ | —CH(C₂H₅)— |
| 430 | C₂H₅ | CH₂CH₂CH₂CH₂CH₂CH₃ | —CH(C₂H₅)— |
| 431 | C₂H₅ | cyclopentyl | —CH(C₂H₅)— |
| 432 | C₂H₅ | cyclohexyl | —CH(C₂H₅)— |
| 433 | C₂H₅ | allyl | —CH(C₂H₅)— |
| 434 | C₂H₅ | but-2-en-1-yl | —CH(C₂H₅)— |
| 435 | C₂H₅ | 4-chlorobut-2-en-1-yl | —CH(C₂H₅)— |
| 436 | C₂H₅ | propargyl | —CH(C₂H₅)— |
| 437 | C₂H₅ | C₆H₅ | —CH(C₂H₅)— |
| 438 | C₂H₅ | C₆H₅CH₂ | —CH(C₂H₅)— |
| 439 | C₂H₅ | 2-phenyleth-1-yl | —CH(C₂H₅)— |
| 440 | C₂H₅ | 4-Cl—C₆H₄ | —CH(C₂H₅)— |
| 441 | C₂H₅ | 4-F—C₆H₄ | —CH(C₂H₅)— |
| 442 | C₂H₅ | CH₃ | —C(CH₃)₂— |
| 443 | C₂H₅ | C₂H₅ | —C(CH₃)₂— |
| 444 | C₂H₅ | CH₂CH₂CH₃ | —C(CH₃)₂— |
| 445 | C₂H₅ | CH(CH₃)₂ | —C(CH₃)₂— |
| 446 | C₂H₅ | CH₂CH₂CH₂CH₃ | —C(CH₃)₂— |
| 447 | C₂H₅ | i-C₄H₉ | —C(CH₃)₂— |
| 448 | C₂H₅ | s-C₄H₉ | —C(CH₃)₂— |
| 449 | C₂H₅ | C(CH₃)₃ | —C(CH₃)₂— |
| 450 | C₂H₅ | CH₂CH₂CH₂CH₂CH₃ | —C(CH₃)₂— |
| 451 | C₂H₅ | CH₂CH₂CH₂CH₂CH₂CH₃ | —C(CH₃)₂— |
| 452 | C₂H₅ | cyclopentyl | —C(CH₃)₂— |
| 453 | C₂H₅ | cyclohexyl | —C(CH₃)₂— |
| 454 | C₂H₅ | allyl | —C(CH₃)₂— |
| 455 | C₂H₅ | but-2-en-1-yl | —C(CH₃)₂— |
| 456 | C₂H₅ | 4-chlorobut-2-en-1-yl | —C(CH₃)₂— |
| 457 | C₂H₅ | propargyl | —C(CH₃)₂— |
| 458 | C₂H₅ | C₆H₅ | —C(CH₃)₂— |
| 459 | C₂H₅ | C₆H₅CH₂ | —C(CH₃)₂— |
| 460 | C₂H₅ | 2-phenyleth-1-yl | —C(CH₃)₂— |
| 461 | C₂H₅ | 4-Cl—C₆H₄ | —C(CH₃)₂— |
| 462 | C₂H₅ | 4-F—C₆H₄ | —C(CH₃)₂— |
| 463 | C₂H₅ | CH₃ | —CH₂CH₂— |
| 464 | C₂H₅ | C₂H₅ | —CH₂CH₂— |
| 465 | C₂H₅ | CH₂CH₂CH₃ | —CH₂CH₂— |
| 466 | C₂H₅ | CH(CH₃)₂ | —CH₂CH₂— |
| 467 | C₂H₅ | CH₂CH₂CH₂CH₃ | —CH₂CH₂— |
| 468 | C₂H₅ | i-C₄H₉ | —CH₂CH₂— |
| 469 | C₂H₅ | s-C₄H₉ | —CH₂CH₂— |
| 470 | C₂H₅ | C(CH₃)₃ | —CH₂CH₂— |
| 471 | C₂H₅ | CH₂CH₂CH₂CH₂CH₃ | —CH₂CH₂— |
| 472 | C₂H₅ | CH₂CH₂CH₂CH₂CH₂CH₃ | —CH₂CH₂— |
| 473 | C₂H₅ | cyclopentyl | —CH₂CH₂— |
| 474 | C₂H₅ | cyclohexyl | —CH₂CH₂— |
| 475 | C₂H₅ | allyl | —CH₂CH₂— |
| 476 | C₂H₅ | but-2-en-1-yl | —CH₂CH₂— |
| 477 | C₂H₅ | 4-chlorobut-2-en-1-yl | —CH₂CH₂— |
| 478 | C₂H₅ | propargyl | —CH₂CH₂— |
| 479 | C₂H₅ | C₆H₅ | —CH₂CH₂— |
| 480 | C₂H₅ | C₆H₅CH₂ | —CH₂CH₂— |
| 481 | C₂H₅ | 2-phenyleth-1-yl | —CH₂CH₂— |
| 482 | C₂H₅ | 4-Cl—C₆H₄ | —CH₂CH₂— |
| 483 | C₂H₅ | 4-F—C₆H₄ | —CH₂CH₂— |
| 484 | C₂H₅ | CH₃ | —CH(CH₃)CH₂— |
| 485 | C₂H₅ | C₂H₅ | —CH(CH₃)CH₂— |
| 486 | C₂H₅ | CH₂CH₂CH₃ | —CH(CH₃)CH₂— |
| 487 | C₂H₅ | CH(CH₃)₂ | —CH(CH₃)CH₂— |
| 488 | C₂H₅ | CH₂CH₂CH₂CH₃ | —CH(CH₃)CH₂— |
| 489 | C₂H₅ | i-C₄H₉ | —CH(CH₃)CH₂— |
| 490 | C₂H₅ | s-C₄H₉ | —CH(CH₃)CH₂— |
| 491 | C₂H₅ | C(CH₃)₃ | —CH(CH₃)CH₂— |
| 492 | C₂H₅ | CH₂CH₂CH₂CH₂CH₃ | —CH(CH₃)CH₂— |
| 493 | C₂H₅ | CH₂CH₂CH₂CH₂CH₂CH₃ | —CH(CH₃)CH₂— |
| 494 | C₂H₅ | cyclopentyl | —CH(CH₃)CH₂— |
| 495 | C₂H₅ | cyclohexyl | —CH(CH₃)CH₂— |
| 496 | C₂H₅ | allyl | —CH(CH₃)CH₂— |
| 497 | C₂H₅ | but-2-en-1-yl | —CH(CH₃)CH₂— |
| 498 | C₂H₅ | 4-chlorobut-2-en-1-yl | —CH(CH₃)CH₂— |
| 499 | C₂H₅ | propargyl | —CH(CH₃)CH₂— |
| 500 | C₂H₅ | C₆H₅ | —CH(CH₃)CH₂— |
| 501 | C₂H₅ | C₆H₅CH₂ | —CH(CH₃)CH₂— |
| 502 | C₂H₅ | 2-phenyleth-1-yl | —CH(CH₃)CH₂— |
| 503 | C₂H₅ | 4-Cl—C₆H₄ | —CH(CH₃)CH₂— |
| 504 | C₂H₅ | 4-F—C₆H₄ | —CH(CH₃)CH₂— |
| 505 | C₂H₅ | CH₃ | —CH₂CH(CH₃)— |
| 506 | C₂H₅ | C₂H₅ | —CH₂CH(CH₃)— |
| 507 | C₂H₅ | CH₂CH₂CH₃ | —CH₂CH(CH₃)— |
| 508 | C₂H₅ | CH(CH₃)₂ | —CH₂CH(CH₃)— |
| 509 | C₂H₅ | CH₂CH₂CH₂CH₃ | —CH₂CH(CH₃)— |
| 510 | C₂H₅ | i-C₄H₉ | —CH₂CH(CH₃)— |
| 511 | C₂H₅ | s-C₄H₉ | —CH₂CH(CH₃)— |
| 512 | C₂H₅ | C(CH₃)₃ | —CH₂CH(CH₃)— |
| 513 | C₂H₅ | CH₂CH₂CH₂CH₂CH₃ | —CH₂CH(CH₃)— |
| 514 | C₂H₅ | CH₂CH₂CH₂CH₂CH₂CH₃ | —CH₂CH(CH₃)— |
| 515 | C₂H₅ | cyclopentyl | —CH₂CH(CH₃)— |
| 516 | C₂H₅ | cyclohexyl | —CH₂CH(CH₃)— |
| 517 | C₂H₅ | allyl | —CH₂CH(CH₃)— |
| 518 | C₂H₅ | but-2-en-1-yl | —CH₂CH(CH₃)— |
| 519 | C₂H₅ | 4-chlorobut-2-en-1-yl | —CH₂CH(CH₃)— |
| 520 | C₂H₅ | propargyl | —CH₂CH(CH₃)— |
| 521 | C₂H₅ | C₆H₅ | —CH₂CH(CH₃)— |
| 522 | C₂H₅ | C₆H₅CH₂ | —CH₂CH(CH₃)— |
| 523 | C₂H₅ | 2-phenyleth-1-yl | —CH₂CH(CH₃)— |
| 524 | C₂H₅ | 4-Cl—C₆H₄ | —CH₂CH(CH₃)— |
| 525 | C₂H₅ | 4-F—C₆H₄ | —CH₂CH(CH₃)— |
| 526 | C₂H₅ | CH₃ | —CH(CH₃)CH(CH₃)— |
| 527 | C₂H₅ | C₂H₅ | —CH(CH₃)CH(CH₃)— |
| 528 | C₂H₅ | CH₂CH₂CH₃ | —CH(CH₃)CH(CH₃)— |
| 529 | C₂H₅ | CH(CH₃)₂ | —CH(CH₃)CH(CH₃)— |
| 530 | C₂H₅ | CH₂CH₂CH₂CH₃ | —CH(CH₃)CH(CH₃)— |
| 531 | C₂H₅ | i-C₄H₉ | —CH(CH₃)CH(CH₃)— |
| 532 | C₂H₅ | s-C₄H₉ | —CH(CH₃)CH(CH₃)— |
| 533 | C₂H₅ | C(CH₃)₃ | —CH(CH₃)CH(CH₃)— |
| 534 | C₂H₅ | CH₂CH₂CH₂CH₂CH₃ | —CH(CH₃)CH(CH₃)— |
| 535 | C₂H₅ | CH₂CH₂CH₂CH₂CH₂CH₃ | —CH(CH₃)CH(CH₃)— |
| 536 | C₂H₅ | cyclopentyl | —CH(CH₃)CH(CH₃)— |
| 537 | C₂H₅ | cyclohexyl | —CH(CH₃)CH(CH₃)— |
| 538 | C₂H₅ | allyl | —CH(CH₃)CH(CH₃)— |
| 539 | C₂H₅ | but-2-en-1-yl | —CH(CH₃)CH(CH₃)— |
| 540 | C₂H₅ | 4-chlorobut-2-en-1-yl | —CH(CH₃)CH(CH₃)— |
| 541 | C₂H₅ | propargyl | —CH(CH₃)CH(CH₃)— |
| 542 | C₂H₅ | C₆H₅ | —CH(CH₃)CH(CH₃)— |
| 543 | C₂H₅ | C₆H₅CH₂ | —CH(CH₃)CH(CH₃)— |
| 544 | C₂H₅ | 2-phenyleth-1-yl | —CH(CH₃)CH(CH₃)— |
| 545 | C₂H₅ | 4-Cl—C₆H₄ | —CH(CH₃)CH(CH₃)— |
| 546 | C₂H₅ | 4-F—C₆H₄ | —CH(CH₃)CH(CH₃)— |
| 547 | C₂H₅ | CH₃ | —CH₂CH₂CH₂— |
| 548 | C₂H₅ | C₂H₅ | —CH₂CH₂CH₂— |
| 549 | C₂H₅ | CH₂CH₂CH₃ | —CH₂CH₂CH₂— |
| 550 | C₂H₅ | CH(CH₃)₂ | —CH₂CH₂CH₂— |
| 551 | C₂H₅ | CH₂CH₂CH₂CH₃ | —CH₂CH₂CH₂— |
| 552 | C₂H₅ | i-C₄H₉ | —CH₂CH₂CH₂— |
| 553 | C₂H₅ | s-C₄H₉ | —CH₂CH₂CH₂— |
| 554 | C₂H₅ | C(CH₃)₃ | —CH₂CH₂CH₂— |
| 555 | C₂H₅ | CH₂CH₂CH₂CH₂CH₃ | —CH₂CH₂CH₂— |
| 556 | C₂H₅ | CH₂CH₂CH₂CH₂CH₂CH₃ | —CH₂CH₂CH₂— |
| 557 | C₂H₅ | cyclopentyl | —CH₂CH₂CH₂— |
| 558 | C₂H₅ | cyclohexyl | —CH₂CH₂CH₂— |
| 559 | C₂H₅ | allyl | —CH₂CH₂CH₂— |
| 560 | C₂H₅ | but-2-en-1-yl | —CH₂CH₂CH₂— |
| 561 | C₂H₅ | 4-chlorobut-2-en-1-yl | —CH₂CH₂CH₂— |
| 562 | C₂H₅ | propargyl | —CH₂CH₂CH₂— |
| 563 | C₂H₅ | C₆H₅ | —CH₂CH₂CH₂— |
| 564 | C₂H₅ | C₆H₅CH₂ | —CH₂CH₂CH₂— |
| 565 | C₂H₅ | 2-phenyleth-1-yl | —CH₂CH₂CH₂— |
| 566 | C₂H₅ | 4-Cl—C₆H₄ | —CH₂CH₂CH₂— |
| 567 | C₂H₅ | 4-F—C₆H₄ | —CH₂CH₂CH₂— |
| 568 | CH₂CH₂CH₃ | CH₃ | —CH₂— |
| 569 | CH₂CH₂CH₃ | C₂H₅ | —CH₂— |
| 570 | CH₂CH₂CH₃ | CH₂CH₂CH₃ | —CH₂— |
| 571 | CH₂CH₂CH₃ | CH(CH₃)₂ | —CH₂— |
| 572 | CH₂CH₂CH₃ | CH₂CH₂CH₂CH₃ | —CH₂— |
| 573 | CH₂CH₂CH₃ | i-C₄H₉ | —CH₂— |
| 574 | CH₂CH₂CH₃ | s-C₄H₉ | —CH₂— |
| 575 | CH₂CH₂CH₃ | C(CH₃)₃ | —CH₂— |
| 576 | CH₂CH₂CH₃ | CH₂CH₂CH₂CH₂CH₃ | —CH₂— |
| 577 | CH₂CH₂CH₃ | CH₂CH₂CH₂CH₂CH₂CH₃ | —CH₂— |
| 578 | CH₂CH₂CH₃ | cyclopentyl | —CH₂— |
| 579 | CH₂CH₂CH₃ | cyclohexyl | —CH₂— |
| 580 | CH₂CH₂CH₃ | allyl | —CH₂— |
| 581 | CH₂CH₂CH₃ | but-2-en-1-yl | —CH₂— |
| 582 | CH₂CH₂CH₃ | 4-chlorobut-2-en-1-yl | —CH₂— |
| 583 | CH₂CH₂CH₃ | propargyl | —CH₂— |
| 584 | CH₂CH₂CH₃ | C₆H₅ | —CH₂— |
| 585 | CH₂CH₂CH₃ | C₆H₅CH₂ | —CH₂— |
| 586 | CH₂CH₂CH₃ | 2-phenyleth-1-yl | —CH₂— |
| 587 | CH₂CH₂CH₃ | 4-Cl—C₆H₄ | —CH₂— |
| 588 | CH₂CH₂CH₃ | 4-F—C₆H₄ | —CH₂— |
| 589 | CH₂CH₂CH₃ | CH₃ | —CH(CH₃)— |
| 590 | CH₂CH₂CH₃ | C₂H₅ | —CH(CH₃)— |
| 591 | CH₂CH₂CH₃ | CH₂CH₂CH₃ | —CH(CH₃)— |
| 592 | CH₂CH₂CH₃ | CH(CH₃)₂ | —CH(CH₃)— |
| 593 | CH₂CH₂CH₃ | CH₂CH₂CH₂CH₃ | —CH(CH₃)— |
| 594 | CH₂CH₂CH₃ | i-C₄H₉ | —CH(CH₃)— |
| 595 | CH₂CH₂CH₃ | s-C₄H₉ | —CH(CH₃)— |
| 596 | CH₂CH₂CH₃ | C(CH₃)₃ | —CH(CH₃)— |
| 597 | CH₂CH₂CH₃ | CH₂CH₂CH₂CH₂CH₃ | —CH(CH₃)— |
| 598 | CH₂CH₂CH₃ | CH₂CH₂CH₂CH₂CH₂CH₃ | —CH(CH₃)— |
| 599 | CH₂CH₂CH₃ | cyclopentyl | —CH(CH₃)— |
| 600 | CH₂CH₂CH₃ | cyclohexyl | —CH(CH₃)— |
| 601 | CH₂CH₂CH₃ | allyl | —CH(CH₃)— |
| 602 | CH₂CH₂CH₃ | but-2-en-1-yl | —CH(CH₃)— |
| 603 | CH₂CH₂CH₃ | 4-chlorobut-2-en-1-yl | —CH(CH₃)— |
| 604 | CH₂CH₂CH₃ | propargyl | —CH(CH₃)— |
| 605 | CH₂CH₂CH₃ | C₆H₅ | —CH(CH₃)— |
| 606 | CH₂CH₂CH₃ | C₆H₅CH₂ | —CH(CH₃)— |
| 607 | CH₂CH₂CH₃ | 2-phenyleth-1-yl | —CH(CH₃)— |
| 608 | CH₂CH₂CH₃ | 4-Cl—C₆H₄ | —CH(CH₃)— |
| 609 | CH₂CH₂CH₃ | 4-F—C₆H₄ | —CH(CH₃)— |
| 610 | CH₂CH₂CH₃ | CH₃ | —CH₂CH₂— |
| 611 | CH₂CH₂CH₃ | C₂H₅ | —CH₂CH₂— |
| 612 | CH₂CH₂CH₃ | CH₂CH₂CH₃ | —CH₂CH₂— |
| 613 | CH₂CH₂CH₃ | CH(CH₃)₂ | —CH₂CH₂— |
| 614 | CH₂CH₂CH₃ | CH₂CH₂CH₂CH₃ | —CH₂CH₂— |
| 615 | CH₂CH₂CH₃ | i-C₄H₉ | —CH₂CH₂— |
| 616 | CH₂CH₂CH₃ | s-C₄H₉ | —CH₂CH₂— |
| 617 | CH₂CH₂CH₃ | C(CH₃)₃ | —CH₂CH₂— |
| 618 | CH₂CH₂CH₃ | CH₂CH₂CH₂CH₂CH₃ | —CH₂CH₂— |
| 619 | CH₂CH₂CH₃ | CH₂CH₂CH₂CH₂CH₂CH₃ | —CH₂CH₂— |
| 620 | CH₂CH₂CH₃ | cyclopentyl | —CH₂CH₂— |
| 621 | CH₂CH₂CH₃ | cyclohexyl | —CH₂CH₂— |
| 622 | CH₂CH₂CH₃ | allyl | —CH₂CH₂— |
| 623 | CH₂CH₂CH₃ | but-2-en-1-yl | —CH₂CH₂— |
| 624 | CH₂CH₂CH₃ | 4-chlorobut-2-en-1-yl | —CH₂CH₂— |
| 625 | CH₂CH₂CH₃ | propargyl | —CH₂CH₂— |
| 626 | CH₂CH₂CH₃ | C₆H₅ | —CH₂CH₂— |
| 627 | CH₂CH₂CH₃ | C₆H₅CH₂ | —CH₂CH₂— |
| 628 | CH₂CH₂CH₃ | 2-phenyleth-1-yl | —CH₂CH₂— |
| 629 | CH₂CH₂CH₃ | 4-Cl—C₆H₄ | —CH₂CH₂— |
| 630 | CH₂CH₂CH₃ | 4-F—C₆H₄ | —CH₂CH₂— |
| 631 | CH₂CH₂CH₃ | CH₃ | —CH₂CH₂CH₂— |
| 632 | CH₂CH₂CH₃ | C₂H₅ | —CH₂CH₂CH₂— |
| 633 | CH₂CH₂CH₃ | CH₂CH₂CH₃ | —CH₂CH₂CH₂— |
| 634 | CH₂CH₂CH₃ | CH(CH₃)₂ | —CH₂CH₂CH₂— |
| 635 | CH₂CH₂CH₃ | CH₂CH₂CH₂CH₃ | —CH₂CH₂CH₂— |
| 636 | CH₂CH₂CH₃ | i-C₄H₉ | —CH₂CH₂CH₂— |
| 637 | CH₂CH₂CH₃ | s-C₄H₉ | —CH₂CH₂CH₂— |
| 638 | CH₂CH₂CH₃ | C(CH₃)₃ | —CH₂CH₂CH₂— |
| 639 | CH₂CH₂CH₃ | CH₂CH₂CH₂CH₂CH₃ | —CH₂CH₂CH₂— |
| 640 | CH₂CH₂CH₃ | CH₂CH₂CH₂CH₂CH₂CH₃ | —CH₂CH₂CH₂— |
| 641 | CH₂CH₂CH₃ | cyclopentyl | —CH₂CH₂CH₂— |
| 642 | CH₂CH₂CH₃ | cyclohexyl | —CH₂CH₂CH₂— |
| 643 | CH₂CH₂CH₃ | allyl | —CH₂CH₂CH₂— |
| 644 | CH₂CH₂CH₃ | but-2-en-1-yl | —CH₂CH₂CH₂— |
| 645 | CH₂CH₂CH₃ | 4-chlorobut-2-en-1-yl | —CH₂CH₂CH₂— |
| 646 | CH₂CH₂CH₃ | propargyl | —CH₂CH₂CH₂— |
| 647 | CH₂CH₂CH₃ | C₆H₅ | —CH₂CH₂CH₂— |
| 648 | CH₂CH₂CH₃ | C₆H₅CH₂ | —CH₂CH₂CH₂— |
| 649 | CH₂CH₂CH₃ | 2-phenyleth-1-yl | —CH₂CH₂CH₂— |
| 650 | CH₂CH₂CH₃ | 4-Cl—C₆H₄ | —CH₂CH₂CH₂— |
| 651 | CH₂CH₂CH₃ | 4-F—C₆H₄ | —CH₂CH₂CH₂— |
| 652 | CH(CH₃)₂ | CH₃ | —CH₂— |
| 653 | CH(CH₃)₂ | C₂H₅ | —CH₂— |
| 654 | CH(CH₃)₂ | CH₂CH₂CH₃ | —CH₂— |
| 655 | CH(CH₃)₂ | CH(CH₃)₂ | —CH₂— |
| 656 | CH(CH₃)₂ | CH₂CH₂CH₂CH₃ | —CH₂— |
| 657 | CH(CH₃)₂ | i-C₄H₉ | —CH₂— |
| 658 | CH(CH₃)₂ | s-C₄H₉ | —CH₂— |
| 659 | CH(CH₃)₂ | C(CH₃)₃ | —CH₂— |
| 660 | CH(CH₃)₂ | CH₂CH₂CH₂CH₂CH₃ | —CH₂— |
| 661 | CH(CH₃)₂ | CH₂CH₂CH₂CH₂CH₂CH₃ | —CH₂— |
| 662 | CH(CH₃)₂ | cyclopentyl | —CH₂— |
| 663 | CH(CH₃)₂ | cyclohexyl | —CH₂— |
| 664 | CH(CH₃)₂ | allyl | —CH₂— |
| 665 | CH(CH₃)₂ | but-2-en-1-yl | —CH₂— |
| 666 | CH(CH₃)₂ | 4-chlorobut-2-en-1-yl | —CH₂— |
| 667 | CH(CH₃)₂ | propargyl | —CH₂— |
| 668 | CH(CH₃)₂ | C₆H₅ | —CH₂— |
| 669 | CH(CH₃)₂ | C₆H₅CH₂ | —CH₂— |
| 670 | CH(CH₃)₂ | 2-phenyleth-1-yl | —CH₂— |
| 671 | CH(CH₃)₂ | 4-Cl—C₆H₄ | —CH₂— |
| 672 | CH(CH₃)₂ | 4-F—C₆H₄ | —CH₂— |
| 673 | CH(CH₃)₂ | CH₃ | —CH(CH₃)— |
| 674 | CH(CH₃)₂ | C₂H₅ | —CH(CH₃)— |
| 675 | CH(CH₃)₂ | CH₂CH₂CH₃ | —CH(CH₃)— |
| 676 | CH(CH₃)₂ | CH(CH₃)₂ | —CH(CH₃)— |
| 677 | CH(CH₃)₂ | CH₂CH₂CH₂CH₃ | —CH(CH₃)— |
| 678 | CH(CH₃)₂ | i-C₄H₉ | —CH(CH₃)— |
| 679 | CH(CH₃)₂ | s-C₄H₉ | —CH(CH₃)— |
| 680 | CH(CH₃)₂ | C(CH₃)₃ | —CH(CH₃)— |
| 681 | CH(CH₃)₂ | CH₂CH₂CH₂CH₂CH₃ | —CH(CH₃)— |
| 682 | CH(CH₃)₂ | CH₂CH₂CH₂CH₂CH₂CH₃ | —CH(CH₃)— |
| 683 | CH(CH₃)₂ | cyclopentyl | —CH(CH₃)— |
| 684 | CH(CH₃)₂ | cyclohexyl | —CH(CH₃)— |
| 685 | CH(CH₃)₂ | allyl | —CH(CH₃)— |
| 686 | CH(CH₃)₂ | but-2-en-1-yl | —CH(CH₃)— |
| 687 | CH(CH₃)₂ | 4-chlorobut-2-en-1-yl | —CH(CH₃)— |
| 688 | CH(CH₃)₂ | propargyl | —CH(CH₃)— |
| 689 | CH(CH₃)₂ | C₆H₅ | —CH(CH₃)— |
| 690 | CH(CH₃)₂ | C₆H₅CH₂ | —CH(CH₃)— |
| 691 | CH(CH₃)₂ | 2-phenyleth-1-yl | —CH(CH₃)— |
| 692 | CH(CH₃)₂ | 4-Cl—C₆H₄ | —CH(CH₃)— |
| 693 | CH(CH₃)₂ | 4-F—C₆H₄ | —CH(CH₃)— |
| 694 | CH(CH₃)₂ | CH₃ | —CH₂CH₂— |
| 695 | CH(CH₃)₂ | C₂H₅ | —CH₂CH₂— |
| 696 | CH(CH₃)₂ | CH₂CH₂CH₃ | —CH₂CH₂— |
| 697 | CH(CH₃)₂ | CH(CH₃)₂ | —CH₂CH₂— |
| 698 | CH(CH₃)₂ | CH₂CH₂CH₂CH₃ | —CH₂CH₂— |
| 699 | CH(CH₃)₂ | i-C₄H₉ | —CH₂CH₂— |
| 700 | CH(CH₃)₂ | s-C₄H₉ | —CH₂CH₂— |
| 701 | CH(CH₃)₂ | C(CH₃)₃ | —CH₂CH₂— |
| 702 | CH(CH₃)₂ | CH₂CH₂CH₂CH₂CH₃ | —CH₂CH₂— |
| 703 | CH(CH₃)₂ | CH₂CH₂CH₂CH₂CH₂CH₃ | —CH₂CH₂— |
| 704 | CH(CH₃)₂ | cyclopentyl | —CH₂CH₂— |
| 705 | CH(CH₃)₂ | cyclohexyl | —CH₂CH₂— |
| 706 | CH(CH₃)₂ | allyl | —CH₂CH₂— |
| 707 | CH(CH₃)₂ | but-2-en-1-yl | —CH₂CH₂— |
| 708 | CH(CH₃)₂ | 4-chlorobut-2-en-1-yl | —CH₂CH₂— |
| 709 | CH(CH₃)₂ | propargyl | —CH₂CH₂— |
| 710 | CH(CH₃)₂ | C₆H₅ | —CH₂CH₂— |
| 711 | CH(CH₃)₂ | C₆H₅CH₂ | —CH₂CH₂— |
| 712 | CH(CH₃)₂ | 2-phenyleth-1-yl | —CH₂CH₂— |
| 713 | CH(CH₃)₂ | 4-Cl—C₆H₄ | —CH₂CH₂— |
| 714 | CH(CH₃)₂ | 4-F—C₆H₄ | —CH₂CH₂— |
| 715 | CH(CH₃)₂ | CH₃ | —CH₂CH₂CH₂— |
| 716 | CH(CH₃)₂ | C₂H₅ | —CH₂CH₂CH₂— |
| 717 | CH(CH₃)₂ | CH₂CH₂CH₃ | —CH₂CH₂CH₂— |
| 718 | CH(CH₃)₂ | CH(CH₃)₂ | —CH₂CH₂CH₂— |
| 719 | CH(CH₃)₂ | CH₂CH₂CH₂CH₃ | —CH₂CH₂CH₂— |
| 720 | CH(CH₃)₂ | i-C₄H₉ | —CH₂CH₂CH₂— |
| 721 | CH(CH₃)₂ | s-C₄H₉ | —CH₂CH₂CH₂— |
| 722 | CH(CH₃)₂ | C(CH₃)₃ | —CH₂CH₂CH₂— |
| 723 | CH(CH₃)₂ | CH₂CH₂CH₂CH₂CH₃ | —CH₂CH₂CH₂— |
| 724 | CH(CH₃)₂ | CH₂CH₂CH₂CH₂CH₂CH₃ | —CH₂CH₂CH₂— |
| 725 | CH(CH₃)₂ | cyclopentyl | —CH₂CH₂CH₂— |
| 726 | CH(CH₃)₂ | cyclohexyl | —CH₂CH₂CH₂— |
| 727 | CH(CH₃)₂ | allyl | —CH₂CH₂CH₂— |
| 728 | CH(CH₃)₂ | but-2-en-1-yl | —CH₂CH₂CH₂— |
| 729 | CH(CH₃)₂ | 4-chlorobut-2-en-1-yl | —CH₂CH₂CH₂— |
| 730 | CH(CH₃)₂ | propargyl | —CH₂CH₂CH₂— |
| 731 | CH(CH₃)₂ | C₆H₅ | —CH₂CH₂CH₂— |
| 732 | CH(CH₃)₂ | C₆H₅CH₂ | —CH₂CH₂CH₂— |
| 733 | CH(CH₃)₂ | 2-phenyleth-1-yl | —CH₂CH₂CH₂— |
| 734 | CH(CH₃)₂ | 4-Cl—C₆H₄ | —CH₂CH₂CH₂— |
| 735 | CH(CH₃)₂ | 4-F—C₆H₄ | —CH₂CH₂CH₂— |
| 736 | C₆H₅ | CH₃ | —CH₂— |
| 737 | C₆H₅ | C₂H₅ | —CH₂— |
| 738 | C₆H₅ | CH₂CH₂CH₃ | —CH₂— |
| 739 | C₆H₅ | CH(CH₃)₂ | —CH₂— |
| 740 | C₆H₅ | CH₂CH₂CH₂CH₃ | —CH₂— |
| 741 | C₆H₅ | i-C₄H₉ | —CH₂— |
| 742 | C₆H₅ | s-C₄H₉ | —CH₂— |
| 743 | C₆H₅ | C(CH₃)₃ | —CH₂— |
| 744 | C₆H₅ | CH₂CH₂CH₂CH₂CH₃ | —CH₂— |
| 745 | C₆H₅ | CH₂CH₂CH₂CH₂CH₂CH₃ | —CH₂— |
| 746 | C₆H₅ | cyclopentyl | —CH₂— |
| 747 | C₆H₅ | cyclohexyl | —CH₂— |
| 748 | C₆H₅ | allyl | —CH₂— |
| 749 | C₆H₅ | but-2-en-1-yl | —CH₂— |
| 750 | C₆H₅ | 4-chlorobut-2-en-1-yl | —CH₂— |
| 751 | C₆H₅ | propargyl | —CH₂— |
| 752 | C₆H₅ | C₆H₅ | —CH₂— |
| 753 | C₆H₅ | C₆H₅CH₂ | —CH₂— |
| 754 | C₆H₅ | 2-phenyleth-1-yl | —CH₂— |
| 755 | C₆H₅ | 4-Cl—C₆H₄ | —CH₂— |
| 756 | C₆H₅ | 4-F—C₆H₄ | —CH₂— |
| 757 | C₆H₅ | CH₃ | —CH(CH₃)— |
| 758 | C₆H₅ | C₂H₅ | —CH(CH₃)— |
| 759 | C₆H₅ | CH₂CH₂CH₃ | —CH(CH₃)— |
| 760 | C₆H₅ | CH(CH₃)₂ | —CH(CH₃)— |
| 761 | C₆H₅ | CH₂CH₂CH₂CH₃ | —CH(CH₃)— |
| 762 | C₆H₅ | i-C₄H₉ | —CH(CH₃)— |
| 763 | C₆H₅ | s-C₄H₉ | —CH(CH₃)— |
| 764 | C₆H₅ | C(CH₃)₃ | —CH(CH₃)— |
| 765 | C₆H₅ | CH₂CH₂CH₂CH₂CH₃ | —CH(CH₃)— |
| 766 | C₆H₅ | CH₂CH₂CH₂CH₂CH₂CH₃ | —CH(CH₃)— |
| 767 | C₆H₅ | cyclopentyl | —CH(CH₃)— |
| 768 | C₆H₅ | cyclohexyl | —CH(CH₃)— |
| 769 | C₆H₅ | allyl | —CH(CH₃)— |
| 770 | C₆H₅ | but-2-en-1-yl | —CH(CH₃)— |
| 771 | C₆H₅ | 4-chlorobut-2-en-1-yl | —CH(CH₃)— |
| 772 | C₆H₅ | propargyl | —CH(CH₃)— |
| 773 | C₆H₅ | C₆H₅ | —CH(CH₃)— |
| 774 | C₆H₅ | C₆H₅CH₂ | —CH(CH₃)— |
| 775 | C₆H₅ | 2-phenyleth-1-yl | —CH(CH₃)— |
| 776 | C₆H₅ | 4-Cl—C₆H₄ | —CH(CH₃)— |
| 777 | C₆H₅ | 4-F—C₆H₄ | —CH(CH₃)— |
| 778 | C₆H₅ | CH₃ | —CH₂CH₂— |
| 779 | C₆H₅ | C₂H₅ | —CH₂CH₂— |
| 780 | C₆H₅ | CH₂CH₂CH₃ | —CH₂CH₂— |
| 781 | C₆H₅ | CH(CH₃)₂ | —CH₂CH₂— |
| 782 | C₆H₅ | CH₂CH₂CH₂CH₃ | —CH₂CH₂— |
| 783 | C₆H₅ | i-C₄H₉ | —CH₂CH₂— |
| 784 | C₆H₅ | s-C₄H₉ | —CH₂CH₂— |
| 785 | C₆H₅ | C(CH₃)₃ | —CH₂CH₂— |
| 786 | C₆H₅ | CH₂CH₂CH₂CH₂CH₃ | —CH₂CH₂— |
| 787 | C₆H₅ | CH₂CH₂CH₂CH₂CH₂CH₃ | —CH₂CH₂— |
| 788 | C₆H₅ | cyclopentyl | —CH₂CH₂— |
| 789 | C₆H₅ | cyclohexyl | —CH₂CH₂— |
| 790 | C₆H₅ | allyl | —CH₂CH₂— |
| 791 | C₆H₅ | but-2-en-1-yl | —CH₂CH₂— |
| 792 | C₆H₅ | 4-chlorobut-2-en-1-yl | —CH₂CH₂— |
| 793 | C₆H₅ | propargyl | —CH₂CH₂— |
| 794 | C₆H₅ | C₆H₅ | —CH₂CH₂— |
| 795 | C₆H₅ | C₆H₅CH₂ | —CH₂CH₂— |
| 796 | C₆H₅ | 2-phenyleth-1-yl | —CH₂CH₂— |
| 797 | C₆H₅ | 4-Cl—C₆H₄ | —CH₂CH₂— |
| 798 | C₆H₅ | 4-F—C₆H₄ | —CH₂CH₂— |
| 799 | C₆H₅ | CH₃ | —CH₂CH₂CH₂— |
| 800 | C₆H₅ | C₂H₅ | —CH₂CH₂CH₂— |
| 801 | C₆H₅ | CH₂CH₂CH₃ | —CH₂CH₂CH₂— |
| 802 | C₆H₅ | CH(CH₃)₂ | —CH₂CH₂CH₂— |
| 803 | C₆H₅ | CH₂CH₂CH₂CH₃ | —CH₂CH₂CH₂— |
| 804 | C₆H₅ | i-C₄H₉ | —CH₂CH₂CH₂— |
| 805 | C₆H₅ | s-C₄H₉ | —CH₂CH₂CH₂— |
| 806 | C₆H₅ | C(CH₃)₃ | —CH₂CH₂CH₂— |
| 807 | C₆H₅ | CH₂CH₂CH₂CH₂CH₃ | —CH₂CH₂CH₂— |
| 808 | C₆H₅ | CH₂CH₂CH₂CH₂CH₂CH₃ | —CH₂CH₂CH₂— |
| 809 | C₆H₅ | cyclopentyl | —CH₂CH₂CH₂— |
| 810 | C₆H₅ | cyclohexyl | —CH₂CH₂CH₂— |
| 811 | C₆H₅ | allyl | —CH₂CH₂CH₂— |
| 812 | C₆H₅ | but-2-en-1-yl | —CH₂CH₂CH₂— |
| 813 | C₆H₅ | 4-chlorobut-2-en-1-yl | —CH₂CH₂CH₂— |
| 814 | C₆H₅ | propargyl | —CH₂CH₂CH₂— |
| 815 | C₆H₅ | C₆H₅ | —CH₂CH₂CH₂— |
| 816 | C₆H₅ | C₆H₅CH₂ | —CH₂CH₂CH₂— |
| 817 | C₆H₅ | 2-phenyleth-1-yl | —CH₂CH₂CH₂— |
| 818 | C₆H₅ | 4-Cl—C₆H₄ | —CH₂CH₂CH₂— |
| 819 | C₆H₅ | 4-F—C₆H₄ | —CH₂CH₂CH₂— |

s-C₄H₉: —CH(CH₃)(C₂H₅);
i-C₄H₉: CH₂CH(CH₃)₂;
allyl: —CH₂CH═CH₂;
propargyl: —CH₂C≡CH;

Table 1:

Compounds of the formula IA, in which A is 2-chlorophenyl and R⁵, R⁶ and (C(R^{3m})(R^{4m}))ₘ for each individual compound correspond in each case to one row of table A.

Table 2:

Compounds of the formula IA, in which A is 2-trifluoromethylphenyl and R⁵, R⁶ and (C(R^{3m})(R^{4m}))ₘ for each individual compound correspond in each case to one row of table A.

Table 3:

Compounds of the formula IA, in which A is 2-difluoromethylphenyl and R⁵, R⁶ and (C(R^{3m})(R^{4m}))ₘ for each individual compound correspond in each case to one row of table A.

Table 4:

Compounds of the formula IA, in which A is 2-methylphenyl and R⁵, R⁶ and (C(R^{3m})(R^{4m}))ₘ for each individual compound correspond in each case to one row of table A.

Table 5:

Compounds of the formula IA, in which A is 2-chloropyridin-3-yl and R⁵, R⁶ and (C(R^{3m})(R^{4m}))ₘ for each individual compound correspond in each case to one row of table A.

Table 6:

Compounds of the formula IA, in which A is 2-trifluoromethylpyridin-3-yl and R⁵, R⁶ and (C(R^{3m})(R^{4m}))ₘ for each individual compound correspond in each case to one row of table A.

Table 7:

Compounds of the formula IA, in which A is 2-difluoromethylpyridin-3-yl and R⁵, R⁶ and (C(R^{3m})(R^{4m}))ₘ for each individual compound correspond in each case to one row of table A.

Table 8:

Compounds of the formula IA, in which A is 2-methylpyridin-3-yl and R⁵, R⁶ and (C(R^{3m})(R^{4m}))ₘ for each individual compound correspond in each case to one row of table A.

Table 9:

Compounds of the formula IA, in which A is 4-methylpyridimidin-5-yl and R⁵, R⁶ and (C(R^{3m})(R^{4m}))ₘ for each individual compound correspond in each case to one row of table A.

Table 10:

Compounds of the formula IA, in which A is 4-trifluoromethylpyrimidin-5-yl and R⁵, R⁶ and (C(R^{3m})(R^{4m}))ₘ for each individual compound correspond in each case to one row of table A.

Table 11:

Compounds of the formula IA, in which A is 4-difluoromethylpyrimidin-5-yl and R⁵, R⁶ and (C(R^{3m})(R^{4m}))ₘ for each individual compound correspond in each case to one row of table A.

Table 12:

Compounds of the formula IA, in which A is 1-methyl-3-trifluoromethylpyrazol-4-yl and R⁵, R⁶ and (C(R^{3m})(R^{4m}))ₘ for each individual compound correspond in each case to one row of table A.

Table 13:

Compounds of the formula IA, in which A is 1-methyl-3-difluoromethylpyrazol-4-yl and R⁵, R⁶ and (C(R^{3m})(R^{4m}))ₘ for each individual compound correspond in each case to one row of table A.

Table 14:

Compounds of the formula IA, in which A is 1,3-dimethylpyrazol-4-yl and R⁵, R⁶ and (C(R^{3m})(R^{4m}))ₘ for each individual compound correspond in each case to one row of table A.

Table 15:

Compounds of the formula IA, in which A is 1-methyl-3-trifluoromethyl-5-fluoropyrazol-4-yl and R⁵, R⁶ and (C(R^{3m})(R^{4m}))ₘ for each individual compound correspond in each case to one row of table A.

Table 16:

Compounds of the formula IA, in which A is 1-methyl-3-difluoromethyl-5-fluoropyrazol-4-yl and R⁵, R⁶ and (C(R^{3m})(R^{4m}))ₘ for each individual compound correspond in each case to one row of table A.

Table 17:

Compounds of the formula IA, in which A is 1-methyl-3-trifluoromethyl-5-chloropyrazol-4-yl and R⁵, R⁶ and (C(R^{3m})(R^{4m}))ₘ for each individual compound correspond in each case to one row of table A.

Table 18:

Compounds of the formula IA, in which A is 1-methyl-3-trifluoromethylpyrol-4-yl and R⁵, R⁶ and (C(R^{3m})(R^{4m}))ₘ for each individual compound correspond in each case to one row of table A.

Table 19:

Compounds of the formula IA, in which A is 1-methyl-3-difluoromethylpyrol-4-yl and R⁵, R⁶ and (C(R^{3m})(R^{4m}))ₘ for each individual compound correspond in each case to one row of table A.

Table 20:

Compounds of the formula IA, in which A is 2-methyl-4-trifluoromethylthiazol-5-yl and R⁵, R⁶ and (C(R^{3m})(R^{4m}))ₘ for each individual compound correspond in each case to one row of table A.

Table 21:

Compounds of the formula IA, in which A is 2-methyl-4-difluoromethylthiazol-5-yl and R⁵, R⁶ and (C(R^{3m})(R^{4m}))ₘ for each individual compound correspond in each case to one row of table A.

Table 22:

Compounds of the formula IA, in which A is 2,4-dimethylthiazol-5-yl and R⁵, R⁶ and (C(R^{3m})(R^{4m}))ₘ for each individual compound correspond in each case to one row of table A.

Table 23:

Compounds of the formula IA, in which A is 2-methyl-5-trifluoromethylthiazol-4-yl and R⁵, R⁶ and (C(R^{3m})(R^{4m}))ₘ for each individual compound correspond in each case to one row of table A.

Table 24:

Compounds of the formula IA, in which A is 2-methyl-5-difluoromethylthiazol-4-yl and R⁵, R⁶ and (C(R^{3m})(R^{4m}))ₘ for each individual compound correspond in each case to one row of table A.

Table 25:

Compounds of the formula IA, in which A is 2,5-dimethylthiazol-4-yl and R⁵, R⁶ and (C(R^{3m})(R^{4m}))ₘ for each individual compound correspond in each case to one row of table A.

Table 26:

Compounds of the formula IA, in which A is 2-methyl-4-trifluoromethyloxazol-5-yl and R⁵, R⁶ and (C(R^{3m})(R^{4m}))ₘ for each individual compound correspond in each case to one row of table A.

Table 27:

Compounds of the formula IA, in which A is 2-methyl-4-difluoromethyloxazol-5-yl and R⁵, R⁶ and (C(R^{3m})(R^{4m}))ₘ for each individual compound correspond in each case to one row of table A.

Table 28:

Compounds of the formula IA, in which A is 2,4-dimethyloxazol-5-yl and R⁵, R⁶ and (C(R^{3m})(R^{4m}))ₘ for each individual compound correspond in each case to one row of table A.

Table 29:

Compounds of the formula IA, in which A is 2-trifluoromethylthiophen-3-yl and R⁵, R⁶ and (C(R^{3m})(R^{4m}))ₘ for each individual compound correspond in each case to one row of table A.

Table 30:

Compounds of the formula IA, in which A is 5-methyl-2-trifluoromethylthiophen-3-yl and R⁵, R⁶ and (C(R^{3m})(R^{4m}))ₘ for each individual compound correspond in each case to one row of table A.

Table 31:

Compounds of the formula IA, in which A is 2-methylthiophen-3-yl and R⁵, R⁶ and (C(R^{3m})(R^{4m}))ₘ for each individual compound correspond in each case to one row of table A.

Table 32:

Compounds of the formula IA, in which A is 2,5-dimethylthiophen-3-yl and R⁵, R⁶ and (C(R^{3m}(R^{4m}))ₘ for each individual compound correspond in each case to one row of table A.

Table 33:

Compounds of the formula IA, in which A is 3-trifluoromethylthiophen-2-yl and R⁵, R⁶ and (C(R^{3m})(R^{4m}))ₘ for each individual compound correspond in each case to one row of table A.

Table 34:

Compounds of the formula IA, in which A is 3-methylthiophen-2-yl and R⁵, R⁶ and (C(R^{3m})(R^{4m}))ₘ for each individual compound correspond in each case to one row of table A.

Table 35:

Compounds of the formula IA, in which A is 3,5-dimethylthiophen-2-yl and R⁵, R⁶ and (C(R^{3m})(R^{4m}))ₘ for each individual compound correspond in each case to one row of table A.

Table 36:

Compounds of the formula IA, in which A is 5-methyl-3-trifluoromethylthiophen-2-yl and R⁵, R⁶ and (C(R^{3m})(R^{4m}))ₘ for each individual compound correspond in each case to one row of table A.

Table 37:

Compounds of the formula IA, in which A is 2-trifluoromethyfuran-3-yl and R⁵, R⁶ and (C(R^{3m})(R^{4m}))ₘ for each individual compound correspond in each case to one row of table A.

Table 38:

Compounds of the formula IA, in which A is 5-methyl-2-trifluoromethyfuran-3-yl and R⁵, R⁶ and (C(R^{3m})(R^{4m}))ₘ for each individual compound correspond in each case to one row of table A.

Table 39:

Compounds of the formula IA, in which A is 2-methylfuran-3-yl and R⁵, R⁶ and (C(R^{3m})(R^{4m}))ₘ for each individual compound correspond in each case to one row of table A.

Table 40:

Compounds of the formula IA, in which A is 2,5-dimethylfuran-3-yl and R⁵, R⁶ and (C(R^{3m})(R^{4m}))ₘ for each individual compound correspond in each case to one row of table A.

Table 41:

Compounds of the formula IA, in which A is 2-methyl-5,6-dihydro[1,4]oxathiin-3-yl and R⁵, R⁶ and (C(R^{3m})(R^{4m}))ₘ for each individual compound correspond in each case to one row of table A.

Table 42:

Compounds of the formula IA, in which A is 2-methyl-5,6-dihydro-4H-thiopyran-3-yl and R⁵, R⁶ and (C(R^{3m})(R^{4m}))ₘ for each individual compound correspond in each case to one row of table A.

The compounds of the formula I according to the invention can be prepared analogously to processes known per se from the prior art, for example in accordance with scheme 1 by reacting activated (heterocyclyl)carboxylic acid derivatives II with an aniline III [Houben-Weyl: “Methoden der organ. Chemie” [Methods of organic chemistry], Georg-Thieme-Verlag, Stuttgart, New York, 1985, Volume E5, pp. 941-1045]. Activated carboxylic acid derivatives II are, for example, halides, activated esters, anhydrides, azides, e.g. chlorides, fluorides, bromides, para-nitrophenyl esters, pentafluorophenyl esters, N-hydroxysuccinimide esters, hydroxybenzotriazol-1-yl esters. In scheme 1, the radicals A, Y, R¹, R², R^{3m}, R^{4m}, R⁵, R⁶, n and m have the meanings given above, in particular the meanings mentioned as being preferred.

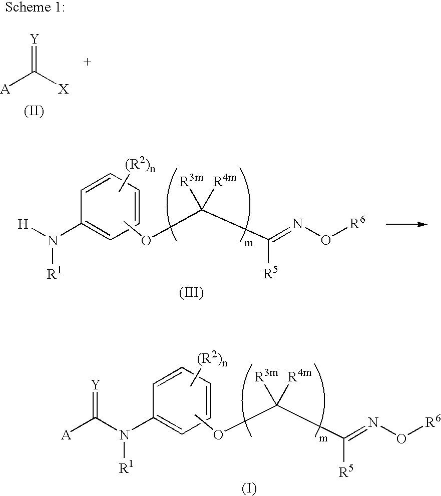

The active compounds I can also be prepared, for example, by reacting the acids IV with an aniline III in the presence of a coupling agent in accordance with scheme 2. In scheme 2, the radicals A, Y, R¹, R², R^{3m}, R^{4m}, R⁵, R⁶, n and m have the meanings mentioned above and in particular the meanings mentioned as being preferred.

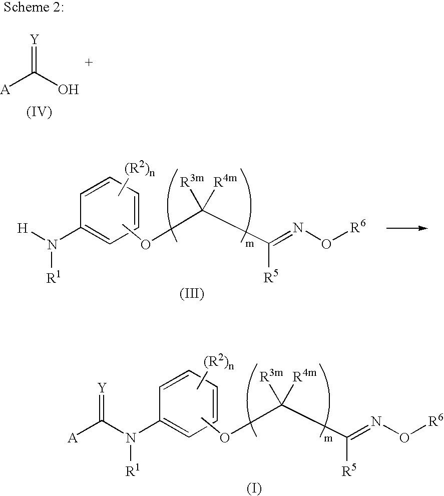

Suitable coupling agents are, for example:
coupling agents based on carbodiimide, for example N,N′-dicyclohexyl-carbodiimide [J. C. Sheehan, G. P. Hess, J. Am. Chem. Soc. 1955, 77, 1067], N-(3-dimethylaminopropyl)-N′-ethylcarbodiimide;
coupling agents which form mixed anhydrides with carbonic esters, for example 2-ethoxy-1-ethoxycarbonyl-1,2-dihydroquinoline [B. Belleau, G. Malek, J. Amer. Chem. Soc. 1968, 90, 1651.], 2-isobutyloxy-1-isobutyloxycarbonyl-1,2-dihydroquinoline [Y. Kiso, H. Yajima, J. Chem. Soc., Chem. Commun. 1972, 942.];
phosphonium-based coupling agents, for example (benzotriazol-1-yloxy)-tris(dimethylamino)phosphonium hexafluorophosphate [B. Castro, J. R. Domoy, G. Evin, C. Selve, Tetrahedron Lett. 1975, 14, 1219.], (benzotriazol-1-yl-oxy)tripyrrolidinophosphonium hexafluorophosphate [J. Coste et. al., Tetrahedron Lett. 1990, 31, 205.];
uronium-based coupling agents or coupling agents having a guanidinium N-oxide structure, for example N,N,N′,N′-tetramethyl-O-(1H-benzotriazol-1-yl)-uronium hexafluorophosphate [R. Knorr, A. Trzeciak, W. Bannwarth, D. Gillessen, Tetrahedron Lett. 1989, 30, 1927.], N,N,N′,N′-tetramethyl-O-(benzotriazol-1-yl)uronium tetrafluoroborate, (benzotriazol-1-yloxy)-dipiperidinocarbenium hexafluorophosphate [S. Chen, J. Xu, Tetrahedron Lett. 1992, 33, 647.];
coupling agents which form acid chlorides, for example bis(2-oxo-3-oxazolidinyl)-phosphinic chloride [J. Diago-Mesequer, Synthesis 1980, 547.].

Compounds I where R¹=unsubstituted or halogen-substituted alkyl or unsubstituted or substituted cycloalkyl can also be prepared by alkylating the amides I (where R¹ is hydrogen and which are obtainable in accordance with scheme 1 or 2) with suitable alkylating agents in the presence of bases, see scheme 3.

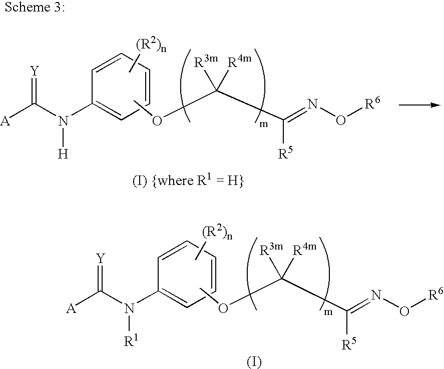

The (heterocyclyl)carboxylic acids IV can be prepared by processes known from the literature, and the (heterocyclyl)carboxylic acid derivatives II are preparable from these compounds by processes known from the literature [for example EP 0589313, EP 915868, U.S. Pat. No. 4,877,441].

The anilines III can be prepared, for example, in accordance with scheme 4. In scheme 4, the radicals R¹, R², R^{3m}, R^{4m}, R⁵, R⁶, n and m have the meanings given above, in particular the meanings mentioned as being preferred. The compounds V and X are known from the literature or can be prepared by processes known from the literature.

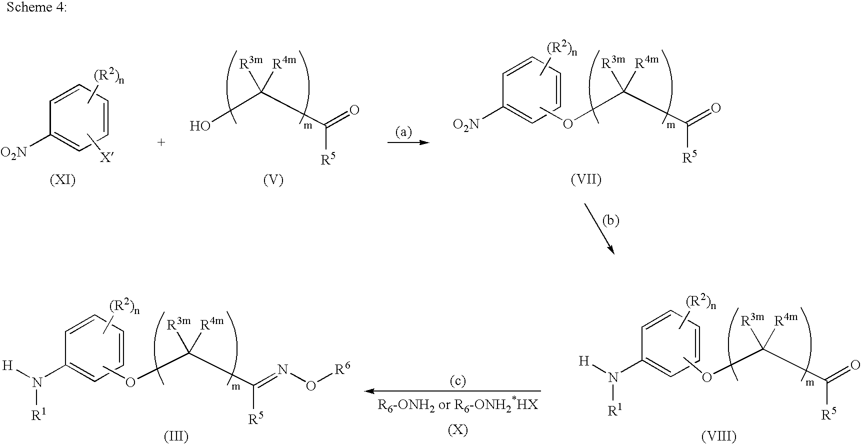

In step a in scheme 4, the nitroaromatic compounds XI in which X′ is a halide, for example chloride or fluoride, is reacted with a keto alcohol V in the sense of a nucleophilic aromatic substitution, giving the nitrophenyl ether VII. The reaction is carried out analogously to known processes, for example according to Organikum, 21st edition, Wiley-VCH 2001, p. 394ff; S. Raeppel, F. Raeppel, J. Suffert; Synlett [SYNLES] 1998, (7), 794-796. R. Beugelmans, A. Bigot, J. Zhu; Tetrahedron Lett [TELEAY] 1994, 35 (31), 5649-5652. The reaction is usually carried out in the presence of a base. Suitable bases are alkali metal carbonates, alkaline earth metal carbonates, such as sodium carbonate, potassium carbonate, calcium carbonate, magnesium carbonate, alkali metal hydroxides or alkaline earth metal hydroxides, such as sodium hydroxide or potassium hydroxide. In general, the reaction is carried out in an inert organic solvent. Suitable solvents are ethers, such as diethyl ether, methyl tert-butyl ether, dioxane, tetrahydrofuran, ethylene glycol dimethyl ether, diethylene glycol.

In step b, the nitrophenyl ether VII is reduced to the aminophenyl ether VIII, for example as described in Organikum, 21st edition, Wiley-VCH 2001, p. 627ff. The catalytic reduction of the nitro group of the nitrophenyl ether VII is generally carried out using hydrazine as hydrogen source, and in the presence of Raney-nickel as catalyst. The reduction is generally carried out in an inert solvent, for example in a C₁-C₄-alcohol, such as methanol or ethanol. The reduction of the nitrophenyl ether VII to the aminophenyl ether VIII can be carried out, for example, by reacting the nitrophenyl ether VII with a metal compound, such as tin(II) chloroide, under acid reaction conditions, such as concentrated hydrochloric acid.

In step c, the aminophenyl ether VIII is reacted with a hydroxylamine X or the acid addition salt thereof, preferably the hydrochloride salt. The reaction is generally carried out in a solvent. Suitable solvents are, for example, C₁-C₄-alcohols or C₁-C₄-alcohol/water mixtures. The reaction can be carried out in the presence of a base. Suitable bases are aromatic amines, such as pyridine, or alkali metal hydroxides or alkaline earth metal hydroxides, such as sodium hydroxide, potassium hydroxide or calcium hydroxide. The oximation of the keto group in X can be carried out, for example, analogously to Organikum, 21st edition, Wiley-VCH 2001, p. 467 or D. Dhanak, C. Reese, S. Romana, G. Zappia, J. Chem. Soc. Chem. Comm. 1986 (12), 903-904, DE 3004871 or AU 580091.

Alternatively, the anilines III can also be prepared in accordance with scheme 5. In scheme 5, the radicals R¹, R², R^{3m}, R^{4m}, R⁵, R⁶, X′, n and m have the meanings given above and in particular the meanings given as being preferred.

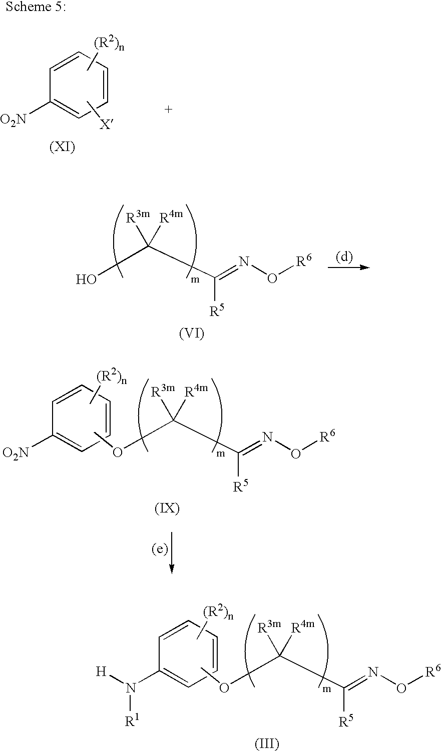

Step d in scheme 5 is carried out analogously to step a in scheme 4. Step e in scheme 5 is carried out analogously to step b in scheme 4.

The oxime IX can also be obtained by reacting the nitrophenyl ether VII with the hydroxylamine X or the acid addition salt of X, analogously to the process described in step a of scheme 4.

The oxime VI can be obtained, for example, by reacting the keto alcohol V with the hydroxylamine X or the acid addition salt of X, analogously to the process described in step a of scheme 4.

The compounds I according to the invention can also be prepared according to scheme 6. In scheme 6, the radicals A, Y, R¹, R², R^{3m}, R^{4m}, R⁵, R⁶, n and m have the meanings given above and in particular the meanings given as being preferred, Hal, Hal′ are independently of one another halogen, for example chloride, bromide or iodide.

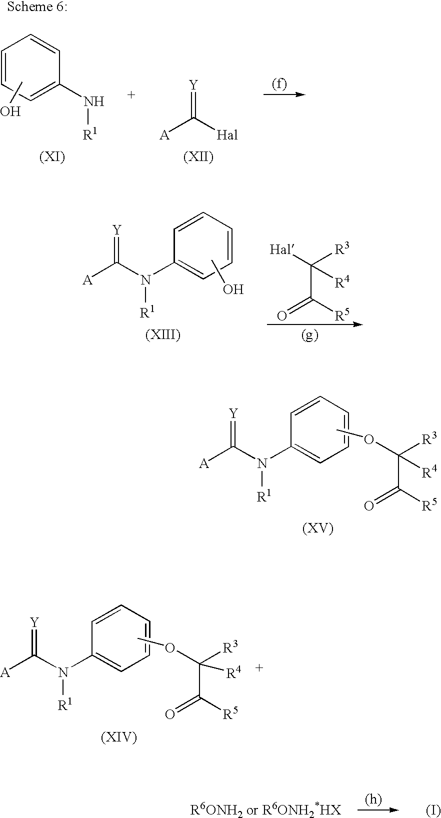

In step f of scheme 6, the aminophenol XI is reacted with a (heterocyclyl)carbonyl halide XII, which affords the anilide XIII. The reaction is usually carried out in the presence of a base, for example a tertiary amine, such as trimethylamine or triethylamine. In general, the reaction is carried out in an inert organic solvent. Suitable solvents are, for example, ethers, such as diethyl ether, methyl tert-butyl ether, dioxane, tetrahydrofuran, ethylene glycol dimethyl ether, diethylene glycol or chlorinated hydrocarbons, such as dichloromethane, dichloroethane or trichloromethane.

The reaction of the anilide XIII with the ketone XIV in step g of scheme 6 can be carried out in the presence of a base. Suitable bases are alkali metal carbonates, alkaline earth metal carbonates, such as sodium carbonate, potassium carbonate, calcium carbonate, magnesium carbonate, alkali metal hydroxides or alkaline earth metal hydroxides, such as sodium hydroxide or potassium hydroxide. In general, the reaction is carried out in an inert organic solvent. Suitable solvents are, for example, carboxamides, such as N,N-dimethylformamide, diethylformamide or dimethylacetamide.

The conversion of the compound XIV into the compound I in step h of scheme 6 is carried out for example analogously to step c of scheme 4.

The compounds I are suitable for use as fungicides. They are distinguished by an outstanding effectiveness against a broad spectrum of phytopathogenic fungi, especially from the classes of the Ascomycetes, Deuteromycetes, Phycomycetes and Basidiomycetes. Some are systemically effective and they can be used in plant protection as foliar and soil fungicides.

They are particularly important in the control of a multitude of fungi on various cultivated plants, such as wheat, rye, barley, oats, rice, maize, grass, bananas, cotton, soya, coffee, sugar cane, vines, fruits and ornamental plants, and vegetables, such as cucumbers, beans, tomatoes, potatoes and cucurbits, and on the seeds of these plants.

They are especially suitable for controlling the following plant diseases:
Alternaria species on fruit and vegetables,
Botrytis cinerea (gray mold) on strawberries, vegetables, ornamental plants and grapevines,
Cercospora arachidicola on groundnuts,
Erysiphe cichoracearum and Sphaerotheca fuliginea on cucurbits,
Erysiphe graminis (powdery mildew) on cereals,
Fusarium and Verticillium species on various plants,
Helminthosporium species on cereals,
Mycosphaerella species on bananas and groundnuts,
Phytophthora infestans on potatoes and tomatoes,
Plasmopara viticola on grapevines,
Podosphaera leucotricha on apples,
Pseudocercosporella herpotrichoides on wheat and barley,
Pseudoperonospora species on hops and cucumbers,
Puccinia species on cereals,
Pyricularia oryzae on rice,
Rhizoctonia species on cotton, rice and lawns,
Septoria nodorum on wheat,
Sphaerotheca fuliginea (mildew of cucumber) on cucumbers,
Uncinula necator on grapevines,
Ustilago species on cereals and sugar cane,
Venturia species (scab) on apples and pears,
Septoria tritici,
Pyrenophora species,
Leptosphaeria nodorum,
Rhynchosporium species and
Typhula species.

The compounds I are also suitable for controlling harmful fungi, such as Paecilomyces variotii, in the protection of materials (e.g. wood, paper, paint dispersions, fibers or fabrics) and in the protection of stored products.

The compounds I are employed by treating the fungi or the plants, seeds, materials or soil to be protected from fungal attack with a fungicidally effective amount of the active compounds. The application can be carried out both before and after the infection of the materials, plants or seeds by the fungi.

The fungicidal compositions generally comprise between 0.1 and 95%, preferably between 0.5 and 90%, by weight of active compound.

When employed in plant protection, the amounts applied are, depending on the kind of effect desired, between 0.01 and 2.0 kg of active compound per ha.

In seed treatment, amounts of active compound of 0.001 to 0.1 g, preferably 0.01 to 0.05 g, per kilogram of seed are generally necessary.

When used in the protection of materials or stored products, the amount of active compound applied depends on the kind of application area and on the effect desired. Amounts customarily applied in the protection of materials are, for example, 0.001 g to 2 kg, preferably 0.005 g to 1 kg, of active compound per cubic meter of treated material.

The compounds I can be converted to the usual formulations, e.g. solutions, emulsions, suspensions, dusts, powders, pastes and granules. The application form depends on the respective use intended; it should in any case guarantee a fine and uniform distribution of the compound according to the invention.

The formulations are prepared in a known way, e.g. by extending the active compound with solvents and/or carriers, if desired using emulsifiers and dispersants, it being possible, when water is the diluent, also to use other organic solvents as auxiliary solvents. Suitable auxiliaries for this purpose are essentially: solvents, such as aromatics (e.g. xylene), chlorinated aromatics (e.g. chlorobenzenes), paraffins (e.g. petroleum fractions), alcohols (e.g. methanol, butanol), ketones (e.g. cyclohexanone), amines (e.g. ethanolamine, dimethylformamide) and water; carriers, such as ground natural minerals (e.g. kaolins, clays, talc, chalk) and ground synthetic ores (e.g. highly dispersed silicic acid, silicates); emulsifiers, such as nonionic and anionic emulsifiers (e.g. polyoxyethylene fatty alcohol ethers, alkylsulfonates and arylsulfonates) and dispersants, such as lignosulfite waste liquors and methylcellulose.

Suitable surfactants are alkali metal, alkaline earth metal and ammonium salts of lignosulfonic acid, naphthalenesulfonic acid, phenolsulfonic acid and dibutylnaphthalenesulfonic acid, alkylarylsulfonates, alkyl sulfates, alkylsulfonates, fatty alcohol sulfates and fatty acids, and alkali metal and alkaline earth metal salts thereof, salts of sulfated fatty alcohol glycol ethers, condensation products of sulfonated naphthalene and naphthalene derivatives with formaldehyde, condensation products of naphthalene or of naphthalenesulfonic acid with phenol and formaldehyde, polyoxyethylene octylphenol ethers, ethoxylated isooctylphenol, octylphenol and nonylphenol, alkylphenol polyglycol ethers, tributylphenyl polyglycol ethers, alkylaryl polyether alcohols, isotridecyl alcohol, fatty alcohol ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

Petroleum fractions having medium to high boiling points, such as kerosene or diesel fuel, furthermore coal tar oils, and oils of vegetable or animal origin, aliphatic, cyclic and aromatic hydrocarbons, e.g. benzene, toluene, xylene, paraffin, tetrahydronaphthalene, alkylated naphthalenes or derivatives thereof, methanol, ethanol, propanol, butanol, chloroform, carbon tetrachloride, cyclohexanol, cyclohexanone, chlorobenzene or isophorone, or highly polar solvents, e.g. dimethylformamide, dimethyl sulfoxide, N-methylpyrrolidone or water, are suitable for the preparation of directly sprayable solutions, emulsions, pastes or oil dispersions.

Powders, combinations for broadcasting and dusts can be prepared by mixing or mutually grinding the active substances with a solid carrier.

Granules, e.g. coated granules, impregnated granules and homogeneous granules, can be prepared by binding the active compounds to solid carriers. Solid carriers are, e.g., mineral earths, such as silica gel, silicic acids, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, e.g., ammonium sulfate, ammonium phosphate, ammonium nitrate or ureas, and plant products, such as cereal meal, tree bark meal, wood meal and nutshell meal, cellulose powders and other solid carriers.

The formulations generally comprise between 0.01 and 95% by weight, preferably between 0.1 and 90% by weight, of the active compound. The active compounds are employed therein in a purity of 90% to 100%, preferably 95% to 100% (according to the NMR spectrum).

Examples for formulations are:
I. 5 parts by weight of a compound according to the invention are intimately mixed with 95 parts by weight of finely divided kaolin. In this way, a dust comprising 5% by weight of the active compound is obtained.
II. 30 parts by weight of a compound according to the invention are intimately mixed with a mixture of 92 parts by weight of pulverulent silica gel and 8 parts by weight of liquid paraffin, which had been sprayed onto the surface of this silica gel. In this way, an active compound preparation with good adhesive properties (active compound content 23% by weight) is obtained.
III. 10 parts by weight of a compound according to the invention are dissolved in a mixture consisting of 90 parts by weight of xylene, 6 parts by weight of the addition product of 8 to 10 mol of ethylene oxide with 1 mol of the N-mono-ethanolamide of oleic acid, 2 parts by weight of the calcium salt of dodecyl-benzenesulfonic acid and 2 parts by weight of the addition product of 40 mol of ethylene oxide with 1 mol of castor oil (active compound content 9% by weight).
IV. 20 parts by weight of a compound according to the invention are dissolved in a mixture consisting of 60 parts by weight of cyclohexanone, 30 parts by weight of isobutanol, 5 parts by weight of the addition product of 7 mol of ethylene oxide with 1 mol of isooctylphenol and 5 parts by weight of the addition product of 40 mol of ethylene oxide with 1 mol of castor oil (active compound content 16% by weight).
V. 80 parts by weight of a compound according to the invention are intimately mixed with 3 parts by weight of the sodium salt of diisobutylnaphthalene-α-sulfonic acid, 10 parts by weight of the sodium salt of a lignosulfonic acid from a sulfite waste liquor and 7 parts by weight of pulverulent silica gel and are ground in a hammer mill (active compound content 80% by weight).
VI. 90 parts by weight of a compound according to the invention are mixed with 10 parts by weight of N-methyl-α-pyrrolidone and a solution is obtained which is suitable for use in the form of very small drops (active compound content 90% by weight).
VII. 20 parts by weight of a compound according to the invention are dissolved in a mixture consisting of 40 parts by weight of cyclohexanone, 30 parts by weight of isobutanol, 20 parts by weight of the adduct of 7 mol of ethylene oxide to 1 mol of isooctylphenol and 10 parts by weight of the adduct of 40 mol of ethylene oxide to 1 mol of castor oil. By running the solution into 100 000 parts by weight of water and finely dispersing it therein, an aqueous dispersion is obtained comprising 0.02% by weight of the active compound.
VIII. 20 parts by weight of a compound according to the invention are intimately mixed with 3 parts by weight of the sodium salt of diisobutylnaphthalene-α-sulfonic acid, 17 parts by weight of the sodium salt of a lignosulfonic acid from a sulfite waste liquor and 60 parts by weight of pulverulent silica gel and are ground in a hammer mill. A spray emulsion comprising 0.1% by weight of the active compound is obtained by fine dispersion of the mixture in 20 000 parts by weight of water.
IX. 10 parts by weight of the compound according to the invention are dissolved in 63 parts by weight of cyclohexanone, 27 parts by weight of dispersing agent (for example a mixture of 50 parts by weight of the adduct of 7 mol of ethylene oxide to 1 mol of isooctylphenol and 50 parts by weight of the adduct of 40 mol of ethylene oxide to 1 mol of castor oil). The stock solution is then diluted to the desired concentration, for example a concentration in the range from 1 to 100 ppm, by distribution in water.

The active compounds can be used as such, in the form of their formulations or of the application forms prepared therefrom, e.g. in the form of directly sprayable solutions, powders, suspensions or dispersions, emulsions, oil dispersions, pastes, dusts, compositions for broadcasting or granules, by spraying, atomizing, dusting, broadcasting or watering. The application forms depend entirely on the intended uses; they should in any case guarantee the finest possible dispersion of the active compounds according to the invention.

Aqueous application forms can be prepared from emulsion concentrates, pastes or wettable powders (spray powders, oil dispersions) by addition of water. To prepare emulsions, pastes or oil dispersions, the substances can be homogenized in water, as such or dissolved in an oil or solvent, by means of wetting agents, tackifiers, dispersants or emulsifiers. However, concentrates comprising active substance, wetting agent, tackifier, dispersant or emulsifier and possibly solvent or oil can also be prepared, which concentrates are suitable for dilution with water.

The concentrations of active compound in the ready-to-use preparations can be varied within relatively wide ranges. In general, they are between 0.0001 and 10%. Often even small amounts of active compound I are sufficient in the ready-to-use preparation, for example 2 to 200 ppm. Ready-to-use preparations with concentrations of active compound in the range from 0.01 to 1% are also preferred.

The active compounds can also be used with great success in the ultra low volume (ULV) process, it being possible to apply formulations with more than 95% by weight of active compound or even the active compound without additives.

Oils of various types, herbicides, fungicides, other pesticides and bactericides can be added to the active compounds, if need be also not until immediately before use (tank mix). These agents can be added to the compositions according to the invention in a weight ratio of 1:10 to 10:1.

The compositions according to the invention can, in the application form as fungicides, also be present together with other active compounds, e.g. with herbicides, insecticides, growth regulators, fungicides or also with fertilizers. On mixing the compounds I or the compositions comprising them in the application form as fungicides with other fungicides, in many cases an expansion of the fungicidal spectrum of activity is obtained.

The following list of fungicides, with which the compounds according to the invention can be used in conjunction, is intended to illustrate the possible combinations but does not limit them:
sulfur, dithiocarbamates and their derivatives, such as iron(III) dimethyldithio-carbamate, zinc dimethyldithiocarbamate, zinc ethylenebisdithiocarbamate, manganese ethylenebisdithiocarbamate, manganese zinc ethylenediaminebisdithiocarbamate, tetramethylthiuram disulfide, ammonia complex of zinc (N,N′-ethylenebisdithiocarbamate), ammonia complex of zinc (N,N′-propylenebisdithiocarbamate), zinc (N,N′-propylenebisdithiocarbamate) or N,N′-poly-propylenebis(thiocarbamoyl)disulfide;
nitro derivatives, such as dinitro(1-methylheptyl)phenyl crotonate, 2-sec-butyl-4,6-dinitrophenyl 3,3-dimethylacrylate, 2-sec-butyl-4,6-dinitrophenyl isopropyl carbonate or diisopropyl 5-nitroisophthalate;
heterocyclic substances, such as 2-heptadecyl-2-imidazoline acetate, 2,4-dichloro-6-(o-chloroanilino)-s-triazine, O,O-diethyl phthalimidophosphono-thioate, 5-amino-1-[bis(dimethylamino)phosphinyl]-3-phenyl-1,2,4-triazole, 2,3-dicyano-1,4-dithioanthraquinone, 2-thio-1,3-dithiolo[4,5-b]quinoxaline, methyl 1-(butylcarbamoyl)-2-benzimidazolecarbamate, 2-(methoxycarbonylamino)benzimidazole, 2-(2-furyl)benzimidazole, 2-(4-thiazolyl)benzimidazole, N-(1,1,2,2-tetrachloroethylthio)tetrahydrophthalimide, N-(trichloromethylthio)tetrahydrophthalimide or N-(trichloromethylthio)phthalimide,
N-dichlorofluoromethylthio-N′,N′-dimethyl-N-phenylsulfdionic acid diamide, 5-ethoxy-3-trichloromethyl-1,2,3-thiadiazole, 2-thiocyanatomethylthiobenzothiazole, 1,4-dichloro-2,5-dimethoxybenzene, 4-(2-chlorophenylhydrazono)-3-methyl-5-isoxazolone, 2-thiopyridine 1-oxide, 8-hydroxyquinoline or its copper salt, 2,3-dihydro-5-carboxanilido-6-methyl-1,4-oxathiin, 2,3-dihydro-5-carboxanilido-6-methyl-1,4-oxathiin 4,4-dioxide, 2-methyl-5,6-dihydro-4H-pyran-3-carboxanilide, 2-methylfuran-3-carboxanilide, 2,5-dimethylfuran-3-carboxanilide, 2,4,5-trimethylfuran-3-carboxanilide, N-cyclohexyl-2,5-dimethylfuran-3-carboxamide, N-cyclohexyl-N-methoxy-2,5-dimethylfuran-3-carboxamide, 2-methylbenzanilide, 2-iodobenzanilide, N-formyl-N-morpholine 2,2,2-trichloroethyl acetal, piperazine-1,4-diylbis-1-(2,2,2-trichloroethyl)formamide, 1-(3,4-dichloroanilino)-1-formylamino-2,2,2-trichloroethane, 2,6-dimethyl-N-tridecylmorpholine or its salts, 2,6-dimethyl-N-cyclododecylmorpholine or its salts, N-[3-(p-(tert-butyl)phenyl)-2-methylpropyl]-cis-2,6-dimethylmorpholine, N-[3-(p-(tert-butyl)phenyl)-2-methyl-propyl]piperidine, 1-[2-(2,4-dichlorophenyl)-4-ethyl-1,3-dioxolan-2-ylethyl]-1H-1,2,4-triazole, 1-[2-(2,4-dichlorophenyl)-4-(n-propyl)-1,3-dioxolan-2-ylethyl]-1H-1,2,4-triazole, N-(n-propyl)-N-(2,4,6-trichlorophenoxyethyl)-N′-imidazolylurea, 1-(4-chlorophenoxy)-3,3-dimethyl-1-(1H-1,2,4-triazol-1-yl)-2-butanone, 1-(4-chlorophenoxy)-3,3-dimethyl-1-(1H-1,2,4-triazol-1-yl)-2-butanol, (2RS,3RS)-1-[3-(2-chlorophenyl)-2-(4-fluorophenyl)oxiran-2-ylmethyl]-1H-1,2,4-triazole, α-(2-chlorophenyl)-α-(4-chlorophenyl)-5-pyrimidine methanol, 5-butyl-2-dimethylamino-4-hydroxy-6-methylpyrimidine, bis(p-chlorophenyl)-3-pyridinemethanol, 1,2-bis(3-ethoxycarbonyl-2-thioureido)benzene or 1,2-bis(3-methoxycarbonyl-2-thioureido)benzene,
strobilurins, such as methyl E-methoxyimino[α-(o-tolyloxy)-o-tolyl]acetate, methyl E-2-{2-[6-(2-cyanophenoxy)pyrimidin-4-yloxy]phenyl}-3-methoxyacrylate, methyl E-methoxyimino-[α-(2-phenoxyphenyl)] acetamide, methyl E-methoxyimino-[α-(2,5-dimethylphenoxy)-o-tolyl]acetamide,
anilinopyrimidines, such as N-(4,6-dimethylpyrimidin-2-yl)aniline, N-[4-methyl-6-(1-propynyl)pyrimidin-2-yl]aniline or N-[4-methyl-6-cyclopropylpyrimidin-2-yl]-aniline,
phenylpyrroles, such as 4-(2,2-difluoro-1,3-benzodioxol-4-yl)pyrrole-3-carbonitrile,
cinnamamides, such as 3-(4-chlorophenyl)-3-(3,4-dimethoxyphenyl)acryloylmorpholine,
and various fungicides, such as dodecylguanidine acetate, 3-[3-(3,5-dimethyl-2-oxycyclohexyl)-2-hydroxyethyl]glutarimide, hexachlorobenzene, methyl N-(2,6-dimethylphenyl)-N-(2-furoyl)-DL-alaninate, N-(2,6-dimethylphenyl)-N-(2′-methoxy-acetyl)-DL-alanine methyl ester, N-(2,6-dimethylphenyl)-N-chloroacetyl-D,L-2-aminobutyrolactone, N-(2,6-dimethylphenyl)-N-(phenylacetyl)-DL-alanine methyl ester, 5-methyl-5-vinyl-3-(3,5-dichlorophenyl)-2,4-dioxo-1,3-oxazolidine, 3-(3,5-dichlorophenyl)-5-methyl-5-methoxymethyl-1,3-oxazolidine-2,4-dione, 3-(3,5-dichlorophenyl)-1-isopropylcarbamoylhydantoin, N-(3,5-dichlorophenyl)-1,2-dimethylcyclopropane-1,2-dicarboximide, 2-cyano-N-(ethylaminocarbonyl)-2-[methoxyimino]acetamide, 1-[2-(2,4-dichlorophenyl)pentyl]-1H-1,2,4-triazole, 2,4-difluoro-α-(1H-1,2,4-triazolyl-1-methyl)benzhydryl alcohol, N-(3-chloro-2,6-dinitro-4-trifluoromethylphenyl)-5-trifluoromethyl-3-chloro-2-aminopyridine, 1-((bis(4-fluorophenyl)methylsilyl)methyl)-1H-1,2,4-triazole.

### PREPARATION EXAMPLES

### Example 1

### 2-Chloro-N-(2-(2-benzyloxyimino-1-methyl-n-propoxy)phenyl)nicotinamide

### 1.1 2-Chloro-N-(2-hydroxyphenyl)nicotinamide

At 10° C., a solution of 21 g of 2-chloronicotinyl chloride in 100 ml of dichloromethane was added to a solution of 13.1 g of ortho-aminophenol and 24.2 g of triethylamine in 200 ml of dichloromethane, and the mixture was stirred at 10° C. for 1 hour and at room temperature for 60 h. The reaction mixture was then concentrated under reduced pressure, and the resulting residue was taken up in ethyl acetate. The organic phase was washed twice with dil. hydrochloric acid and 3% strength aqueous sodium hydroxide solution. After drying over sodium sulfate, the solvent was evaporated under reduced pressure, giving 27.6 g of the title compound of melting point 142-145° C.

### 1.2 2-Chloro-N-(2-(1-methyl-2-oxo-n-propoxy)phenyl)nicotinamide

A solution of 1.24 g of 2-chloro-N-(2-hydroxyphenyl)nicotinamide, 1.58 g of 3-bromobutan-2-one and 0.34 g of potassium carbonate in 20 ml of N,N-dimethylformamide was stirred at room temperature for 1 h and then at 60° C. for 2 h. A mixture of water and ethyl acetate was then added, and the phases were separated. The aqueous phase was extracted twice with ethyl acetate. The combined organic phase was washed with saturated NaCl solution, dried over sodium sulfate and concentrated under reduced pressure. The resulting residue was purified chromatographically on silica gel (mobile phase: cyclohexane/methyl tert-butyl ether), giving, after evaporation of the eluent, 1.0 g of the title compound as an oil.

### 1.3 2-Chloro-N-(2-(2-benzyloxyimino-1-methyl-n-propoxy)phenyl)nicotinamide

0.18 g of O-Benzylhydroxylamine was added to a solution of 0.36 g of 2-chloro-N-(2-(1-methyl-2-oxo-n-propoxy)phenyl)nicotinamide and 0.12 g of pyridine in 10 ml of methanol. The mixture was concentrated at room temperature for 15 minutes, the solvent was evaporated under reduced pressure and the resulting residue was taken up in methyl tert-butyl ether. The mixture was washed with 1% strength hydrochloric acid and sat. NaCl solution and dried over sodium sulfate, and the mixture was concentrated under reduced pressure. The precipitated crystals were filtered off and dried under reduced pressure, giving 0.3 g of the title compound of melting point 53-55° C.

The compounds of the formula IA listed in table 43 were prepared in an analogous manner.

TABLE 43

| | |
|---|---|
| | (IA) |
| 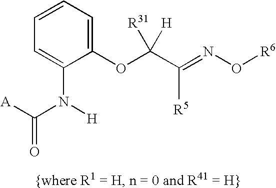 | |

| | | | | | m.p. [° C.]; | |
|---|---|---|---|---|---|---|
| No. | A | R³¹ | R⁵ | R⁶ | consistency | Spectroscopical data |
| IA-1 | 2-chloro- | CH₃ | CH₃ | C₆H₅CH₂ | 53-55 | |
| | pyridin-3-yl | | | | | |
| IA-2 | 2-chloro- | CH₃ | CH₃ | allyl | oil | ¹H-NMR (CDCl₃), δ [ppm]: |
| | pyridin-3-yl | | | | | 1.57 (d, 3H); 1.83 (s, 3H); |
| | | | | | | 4.58 (m, 2H); 5.04 (m, |
| | | | | | | 1H); 5.18-5.31 (m, 2H); |
| | | | | | | 5.93 (m, 1H); 6.99-7.10 |
| | | | | | | (m, 3H); 7.22 (m, 1H); |
| | | | | | | 8.18 (m, 1H); 8.51 (m, |
| | | | | | | 1H); 9.22 (S_{broad}, 1H). |
| IA-3 | 2-chloro- | CH₃ | CH₃ | trans-2- | oil | ¹H-NMR (CDCl₃), δ [ppm]: |
| | pyridin-3-yl | | | buten-1-yl | | 1.57 (d, 3H); 1.70 (m, 3H); |
| | | | | | | 1.80 (s, 3H); 4.49 (m, 2H); |
| | | | | | | 5.02 (q, 1H); 5.58-5.80 (m, |
| | | | | | | 2H); 6.99-7.10 (m, 3H); |
| | | | | | | 7.22 (m, 1H); 8.16 (m, |
| | | | | | | 1H); 8.51 (m, 2H); 9.20 |
| | | | | | | (S_{broad}, 1H). |
| IA-4 | 2-methyl- | CH₃ | CH₃ | CH₃ | oil | ¹H-NMR (CDCl₃), δ [ppm]: |
| | 4-trifluoro- | | | | | 1.52 (d, 3H); 1.77 (s, 3H); |
| | methyl- | | | | | 2.79 (s, 3H); 3.90 (s, 3H); |
| | thiazol-5-yl | | | | | 5.01 (q, 1H); 6.93-7.11 (m, |
| | | | | | | 4H); 8.43 (m, 1H); 8.70 |
| | | | | | | (m, 1H). |
| IA-5 | 2-methyl- | CH₃ | CH₃ | trans-3- | oil | ¹H-NMR (CDCl₃), δ [ppm]: |
| | 4-trifluoro- | | | chloroallyl | | 1.53 (d, 3H); 1.77 (s, 3H); |
| | methyl- | | | | | 2.75 (s, 3H); 4.53 (d, 2H); |
| | thiazol-5-yl | | | | | 5.01 (q, 1H); 6.07 (m, 1H); |
| | | | | | | 6.20-6.33 (m, 1H); |
| | | | | | | 6.93-7.11 (m, 3H); 8.45 (m, |
| | | | | | | 1H); 8.84 (S_{broad}, 1H). |
| IA-6 | 1-methyl- | CH₃ | CH₃ | trans-3- | oil | ¹H-NMR (CDCl₃), δ [ppm]: |
| | 3-trifluoro- | | | chloroallyl | | 1.53 (d, 3H); 1.79 (s, 3H); |
| | methyl- | | | | | 3.95 (s, 3H); 4.54 (d, 2H); |
| | pyrazol-4- | | | | | 5.00 (q, 1H); 6.08 (m, 1H); |
| | yl | | | | | 6.17—6.29 (m, 1H); |
| | | | | | | 6.96-7.10 (m, 2H); 8.10 (m, |
| | | | | | | 1H); 8.45 (m, 1H); 8.59 |
| | | | | | | (S_{broad}, 1H). |
| IA-7 | 1-methyl- | CH₃ | CH₃ | CH₃ | 100-102 | |
| | 3-trifluoro- | | | | | |
| | methyl- | | | | | |
| | pyrazol-4- | | | | | |
| | yl | | | | | |
| IA-8 | 1-methyl- | CH₃ | CH₃ | C₆H₅CH₂ | oil | ¹H-NMR (CDCl₃), δ [ppm]: |
| | 3-trifluoro- | | | | | 1.58 (d, 3H); 1.80 (s, 3H); |
| | methyl- | | | | | 3.95 (s, 3H); 4.98 (m, 1H); |
| | pyrazol-4- | | | | | 5.17 (s, 2H); 6.82-6.99 (m, |
| | yl | | | | | 3H); 7.25-7.45 (m, 4H); |
| | | | | | | 8.07 (m, 1H); 8.46 (m, |
| | | | | | | 1H); 8.59 (S_{broad}, 1H). |
| IA-9 | 2-chloro- | CH₃ | CH₃ | CH(CH₃)₂ | oil | ¹H-NMR (CDCl₃), δ [ppm]: |
| | pyridin-3-yl | | | | | 1.20 (m, 6H); 1.53 (d, 3H); |
| | | | | | | 1.80 (s, 3H); 4.29 (m, 1H); |
| | | | | | | 5.03 (m, 1H); 6.95-7.15 |
| | | | | | | (m, 3H); 7.43 (m, 1H); |
| | | | | | | 8.31 (m, 1H); 8.47-8.51 |
| | | | | | | (m, 2H) ; 9.23 (S_{broad}, 1H). |
| IA-10 | 2-chloro- | CH₃ | CH₃ | trans-3- | oil | ¹H-NMR (CDCl₃), δ [ppm]: |
| | pyridin-3-yl | | | chloroallyl | | 1.57 (d, 3H); 1.80 (s, 3H); |
| | | | | | | 4.52 (d, 2H); 5.01 (q, 1H); |
| | | | | | | 6.09 (m, 1H); 6.18-6.30 |
| | | | | | | (m, 1H); 6.99-7.13 (m, |
| | | | | | | 3H); 7.03 (m, 1H); 8.35 |
| | | | | | | (m, 1H); 8.51 (m, 2H); |
| | | | | | | 9.21 (S_{broad}, 1H). |
| IA-11 | 2-chloro- | CH₃ | CH₃ | CH₃ | 74-75 | |
| | pyridin-3-yl | | | | | |
| IA-12 | 2-chloro- | CH₃ | CH₃ | C₂H₅ | oil | ¹H-NMR (CDCl₃), δ [ppm]: |
| | pyridin-3-yl | | | | | 1.25 (d, 3H); 1.58 (d, 3H); |
| | | | | | | 1.80 (s, 3H); 4.11 (m, 2H); |
| | | | | | | 5.02 (m, 1H); 6.97-7.10 |
| | | | | | | (m, 3H); 7.47 (m, 1H); |
| | | | | | | 8.31 (m, 1H); 8.23-8.28 |
| | | | | | | (m, 2H); 9.22 (S_{broad}, 1H). |

Allyl: CH₂CH═CH₂;
m.p.: Melting point

### Use Examples

The active compounds were prepared as a stock solution comprising 0.25% by weight of active compound in acetone or dimethyl sulfoxide (DMSO). 1% by weight of the emulsifier Uniperol® EL (wetting agent having emulsifying and dispersant action based 10 on ethoxylated alkylphenols) was added to this solution, and the mixture was diluted with water to the desired concentration.

Curative Action Against Brown Rust of Wheat

Leaves of potted wheat seedlings of the cultivar “Kanzler” were dusted with spores of brown rust (Puccinia recondita). The pots were then placed in a chamber with high atmospheric humidity (90 to 95%) at 20-22° C. for 24 hours. During this time, the spores germinated and the germinal tubes penetrated into the leaf tissue. The next day, the infected plants were sprayed to runoff point with an aqueous suspension having the concentration of active compound stated below. The suspensions or emulsions were prepared as described above. After the spray coating had dried on, the test plants were cultivated in a greenhouse at temperatures between 20 and 22° C. and at 65 to 70% relative atmospheric humidity for 7 days. The extent of the rust fungus development on the leaves was then determined.

| No. | Infection at 63 ppm (% of leaf area) |
|---|---|
| IA-4 | 0 |
| IA-5 | 7 |
| IA-7 | 3 |
| untreated | 90 |

Leaves of potted wheat seedlings of the cultivar “Kanzler” were sprayed to runoff point with an aqueous suspension having the concentration of active compound stated below. The next day, the treated plants were dusted with spores of brown rust of wheat (Puccinia recondita). The plants were then placed in a chamber with high atmospheric humidity (90 to 95%), at 20-22° C., for 24 hours. During this time, the spores germinated and the germinal tubes penetrated into the leaf tissue. The next day, the test plants were returned into the greenhouse and cultivated at temperatures between 20 and 22° C. and at 65 to 70% relative atmospheric humidity for a further 7 days. The extent of the rust development on the leaves was then determined visually.

| No. | Infection at 63 ppm (% of leaf area) |
|---|---|
| IA-1 | 10 |
| IA-4 | 3 |
| IA-5 | 3 |
| IA-6 | 5 |
| IA-7 | 3 |
| IA-8 | 3 |
| IA-9 | 5 |
| IA-10 | 5 |
| IA-11 | 10 |
| IA-12 | 3 |
| untreated | 90 |

1. A (hetero)cyclylcarboxanilide of the formula I, 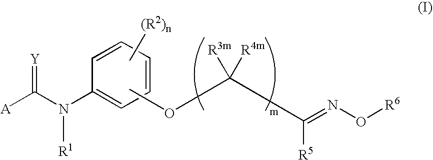
in which variables are as defined below:
A is phenyl or an at least monounsaturated 5- or 6-membered heterocycle having 1, 2 or 3 heteroatoms selected from the group consisting of N, O, S, S(═O) and S(═O)₂ as ring members, where phenyl and the at least monounsaturated 5- or 6-membered heterocycle may be unsubstituted or may carry 1, 2 or 3 radicals R^{a}, where
R^{a} is halogen, nitro, CN, C₁-C₄-alkyl, C₃-C₆-cycloalkyl, C₂-C₄-alkenyl, C₂-C₄-alkynyl, C₁-C₄-alkoxy, C₁-C₄-haloalkyl, C₃-C₆-halocycloalkyl, C₂-C₄-haloalkenyl, C₂-C₄-haloalkynyl, C₁-C₄-haloalkoxy or phenyl, where phenyl may be unsubstituted or carries one, two or three radicals R^{b} selected from the group consisting of halogen, nitro, CN, C₁-C₄-alkyl, C₃-C₆-cycloalkyl, C₂-C₄-alkenyl, C₂-C₄-alkynyl, C₁-C₄-alkoxy, C₁-C₄-haloalkyl, C₃-C₆-halocycloalkyl, C₂-C₄-haloalkenyl, C₂-C₄-haloalkynyl and C₁-C₄-haloalkoxy;
Y is oxygen or sulfur;
R¹ is H, OH, C₁-C₄-alkyl, C₃-C₆-cycloalkyl, C₁-C₄-alkoxy, C₁-C₄-haloalkyl, C₃-C₆-halocycloalkyl or C₁-C₄-haloalkoxy;
R² is halogen, nitro, CN, C₁-C₄-alkyl, C₃-C₆-cycloalkyl, C₂-C₄-alkenyl, C₂-C₄-alkynyl, C₁-C₄-alkoxy, C₁-C₄-haloalkyl, C₃-C₆-halocycloalkyl, C₂-C₄-haloalkenyl, C₂-C₄-haloalkynyl or C₁-C₄-haloalkoxy;
R^{3m}, R^{4m} are each independently of one another halogen, hydrogen, C₁-C₆-alkyl, C₃-C₆-cycloalkyl, C₂-C₆-alkenyl, C₂-C₆-alkynyl, phenyl, phenyl-C₁-C₄-alkyl, phenyl-C₂-C₄-alkenyl, phenyl-C₂-C₄-alkynyl, C₁-C₆-haloalkyl, C₃-C₆-halocycloalkyl, C₂-C₆-haloalkenyl, C₂-C₆-haloalkynyl, phenyl-C₁-C₄-haloalkyl, phenyl-C₂-C₄-haloalkenyl or phenyl-C₂-C₄-haloalkynyl, where phenyl or the phenyl moiety of phenyl-C₁-C₄-alkyl, phenyl-C₂-C₄-alkenyl, phenyl-C₂-C₄-alkynyl, phenyl-C₁-C₄-haloalkyl, phenyl-C₂-C₄-haloalkenyl and phenyl-C₂-C₄-haloalkynyl may be unsubstituted or may carry one, two or three radicals R^{b}; for m=2 or 3 the variables R³², R⁴² and R³³, R⁴³, respectively, may also be C₁-C₆-alkoxy;
R⁵ is hydrogen, C₁-C₆-alkyl, C₃-C₆-cycloalkyl, C₂-C₆-alkenyl, C₂-C₆-alkynyl, phenyl, phenyl-C₁-C₄-alkyl, phenyl-C₂-C₄-alkenyl, phenyl-C₂-C₄-alkynyl, C₁-C₆-haloalkyl, C₃-C₆-halocycloalkyl, C₂-C₆-haloalkenyl, C₂-C₆-haloalkynyl, phenyl-C₁-C₄-haloalkyl, phenyl-C₂-C₄-haloalkenyl or phenyl-C₂-C₄-haloalkynyl, where phenyl or the phenyl moiety of phenyl-C₁-C₄-alkyl, phenyl-C₂-C₄-alkenyl, phenyl-C₂-C₄-alkynyl, phenyl-C₁-C₄-haloalkyl, phenyl-C₂-C₄-haloalkenyl, phenyl-C₂-C₄-haloalkynyl may be unsubstituted or may carry one, two or three radicals R^{b};
R⁶ is hydrogen, C₁-C₈-alkyl, C₃-C₆-cycloalkyl, C₂-C₈-alkenyl, C₂-C₈-alkynyl, C₁-C₈-haloalkyl, C₃-C₆-halocycloalkyl, C₂-C₈-haloalkenyl, C₂-C₈-haloalkynyl, phenyl, naphthyl, phenyl-C₁-C₆-alkyl, naphthyl-C₁-C₆-alkyl, phenyl-C₂-C₆-alkenyl, phenyl-C₂-C₆-alkynyl, phenyl-C₁-C₆-haloalkyl, phenyl-C₂-C₆-haloalkenyl or phenyl-C₂-C₆-haloalkynyl, where phenyl and naphthyl in the 9 last-mentioned groups may be unsubstituted or may carry 1, 2 or 3 substituents selected from the group consisting of R^{b} and R⁷, where R⁷ is —(CR⁸)═NOR⁹, where
R⁸ is hydrogen, C₁-C₆-alkyl, C₃-C₆-cycloalkyl, C₂-C₆-alkenyl, C₂-C₆-alkynyl, C₁-C₆-haloalkyl, C₃-C₆-halocycloalkyl, C₂-C₆-haloalkenyl, C₂-C₆-haloalkynyl, phenyl, benzyl; where phenyl and the phenyl group in benzyl may be unsubstituted or may carry one, two or three radicals R^{b}; and
R⁹ is C₁-C₆-alkyl, C₃-C₆-cycloalkyl, C₂-C₆-alkenyl, C₂-C₆-alkynyl, C₁-C₆-haloalkyl, C₃-C₆-halocycloalkyl, C₂-C₆-haloalkenyl, C₂-C₆-haloalkynyl, phenyl, phenyl-C₁-C₄-alkyl, phenyl-C₁-C₄-haloalkyl, phenyl-C₂-C₄-alkenyl, phenyl-C₂-C₄-haloalkenyl, phenyl-C₂-C₄-alkynyl, phenyl-C₂-C₄-haloalkynyl, where phenyl and the phenyl group in phenyl-C₁-C₄-alkyl, phenyl-C₁-C₄-haloalkyl, phenyl-C₂-C₄-alkenyl, phenyl-C₂-C₄-haloalkenyl, phenyl-C₂-C₄-alkynyl and phenyl-C₂-C₄-haloalkynyl may be unsubstituted or may carry one, two or three radicals R^{b};
n is 0, 1, 2, 3 or 4; and
m is 1, 2 or 3;
or an agriculturally useful salt thereof.

2. A (hetero)cyclylcarboxanilide of the formula I in which A is a radical of the formula 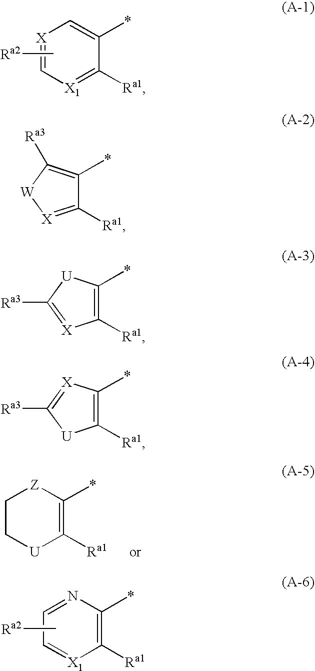
where * means the point of attachment to C(═Y) and the variables are as defined below:
X, X₁ are each independently of one another N or CR^{c}, where R^{c} is H or has one of the meanings mentioned for R^{b};
W is S or N—R^{a4}, where R^{a4} is hydrogen, C₁-C₄-alkyl, C₁-C₄-haloalkyl, C₁-C₄-alkoxy, C₁-C₄-haloalkoxy or phenyl which may be unsubstituted or may carry 1, 2 or 3 radicals R^{b};
U is oxygen or sulfur;
Z is S, S(═O), S(═O)₂ or CH₂,
R^{a1} is hydrogen, C₁-C₄-alkyl, C₁-C₄-alkoxy, C₁-C₄-haloalkyl, C₁-C₄-haloalkoxy or halogen;
R^{a2} are each independently of one another hydrogen, halogen, nitro, CN, C₁-C₄-alkyl, C₃-C₆-cycloalkyl, C₂-C₄-alkenyl, C₂-C₄-alkynyl, C₁-C₄-alkoxy, where the 5 last-mentioned groups may be substituted by halogen; and
R^{a3} is hydrogen, halogen, nitro, CN, C₁-C₄-alkyl, C₃-C₆-cycloalkyl, C₂-C₄-alkenyl, C₂-C₄-alkynyl, C₁-C₄-alkoxy, where the 5 last-mentioned groups may be substituted by halogen.

3. The (hetero)cyclylcarboxanilide of the formula I according to claim 2 in which R^{a1} is hydrogen, halogen, C₁-C₂-alkyl, C₁-C₂-alkoxy or C₁-C₂-fluoroalkyl.

4. The (hetero)cyclylcarboxanilide of the formula I according to claim 2 in which A is a radical of the formula A-1a, A-2a or A-3a, 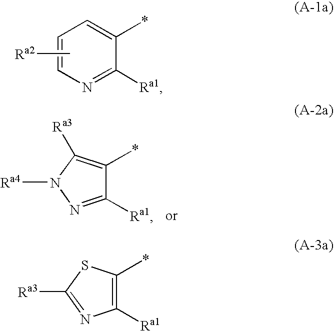
in which R^{a1}, R^{a2}, R^{a3} and R^{a4} are as defined in claim 2.

5. The (hetero)cyclylcarboxanilide of the formula I according to claim 4 in which A is a radical A-1a where R^{a1}=halogen and R^{a2}=hydrogen, or is a radical A-2a where R^{a1} ═C₁-C₂-fluoroalkyl, R^{a3}=is hydrogen and R^{a4}═C₁-C₄-alkyl or is a radical A-3a where R^{a1}═C₁-C₂-fluoroalkyl and R^{a3}═C₁-C₄-alkyl.

6. The (hetero)cyclylcarboxanilide of the formula I according to claim 2 in which R¹ is hydrogen.

7. The (hetero)cyclylcarboxanilide of the formula I according to claim 2 in which R² is C₁-C₄-alkyl, C₁-C₄-alkoxy, C₁-C₄-haloalkyl, C₁-C₄-haloalkoxy, nitro, cyano or halogen.

8. The (hetero)cyclylcarboxanilide of the formula I according to claim 2 in which n is 0 or 1.

9. The (hetero)cyclylcarboxanilide of the formula I according to claim 2 in which m is 1.

10. The (hetero)cyclylcarboxanilide of the formula I according to claim 9 in which R³¹ and R⁴¹ are each independently of one another hydrogen or C₁-C₄-alkyl.

11. The (hetero)cyclylcarboxanilide of the formula I according to claim 2 in which R⁵ is hydrogen, C₁-C₆-alkyl, C₁-C₆-haloalkyl, C₃-C₆-cycloalkyl, C₃-C₆-halocycloalkyl, phenyl, phenyl-C₁-C₄-alkyl, phenyl-C₁-C₄-haloalkyl, where phenyl in the three last-mentioned radicals may be unsubstituted or may carry one, two or three radicals R^{b}.

12. The (hetero)cyclylcarboxanilide of the formula I according to claim 2 in which R⁶ is C₁-C₆-alkyl, C₁-C₆-haloalkyl, C₃-C₆-cycloalkyl, C₃-C₆-halocycloalkyl, C₂-C₆-alkenyl, C₂-C₆-haloalkenyl, C₂-C₄-alkynyl, C₂-C₄-haloalkynyl, phenyl-C₁-C₂-alkyl or phenyl, where phenyl in the two last-mentioned radicals may be unsubstituted or may carry one or two halogen groups.

13. The (hetero)cyclylcarboxanilide of the formula I according to claim 2 in which Y is oxygen.

14. The use of (hetero)cyclylcarboxanilides of the formula I according to claim 2 and of agriculturally useful salts thereof for controlling harmful fungi.

15. A crop protection composition, comprising at least one (hetero)cyclylcarboxanilide of the formula I according to claim 1 or an agriculturally useful salt thereof.

16. A method for controlling harmful fungi, which comprises treating the harmful fungi, their habitat of the plants areas, materials or spaces to be kept free from them with a fungicidally effective amount of at least one (hetero)cyclylcarboxanilide of the formula I according to claim 1 or an agriculturally useful salt thereof.
